(19) United States
(12) Patent Application Publication
(10) Pub. No.: US 2026/0137679 A1
(43) Pub. Date: May 21, 2026

(54) GCN2 INHIBITOR FOR TREATING METASTASES

(71) Applicant: HIBERCELL, INC., New York, NY (US)

(72) Inventors: Nandita BOSE, New York, NY (US); Jeremy DREES, New York, NY (US)

(21) Appl. No.: 19/118,411

(22) PCT Filed: Oct. 4, 2023
(86) PCT No.: PCT/US2023/075995

§ 371 Date: Apr. 4, 2025

Related U.S. Application Data
(60) Provisional application No. 63/413,115, filed on Oct. 4, 2022.

(51) Int. Cl. A61K 31/4985; A61P 35/04

(52) U.S. Cl. CPC A61K 31/4985; A61P 35/04

(57) ABSTRACT

Provided herein are methods of inhibiting myeloid-derived suppressor cells (MDSCs) in a subject in need thereof administering the GCN2 inhibitor of formula (I). The present disclosure also provides methods of treating or reducing the tumor volumes of metastases in a subject in need thereof administering the GCN2 inhibitor of formula (I).

### CROSS-REFERENCE TO RELATED APPLICATIONS

This application claims the benefit of, and priority to, U.S. Provisional Application Ser. No. 63/413,115, filed on Oct. 4, 2022, the contents of which are incorporated by reference herein in their entirety.

### BACKGROUND

Cancer is a leading cause of death worldwide, accounting for nearly 10 million deaths in 2020 (World Health Organization). Targeted therapy and immunotherapy have expanded the horizons for treatment of solid tumors by improving prognosis drastically. However, tumor recurrence, drug resistance, and drug intolerance continue to be major challenges in the management of advanced cancer (Wang et al, “Drug resistance and combating drug resistance in cancer,” Cancer Drug Resistance, 2019, 2(2): 141-160; Chakraborty et al., “The difficulties in cancer treatment” Ecancermedicalscience, 2012, 6:ed16). Cancer cells often experience a variety of stressors in their microenvironment such as hypoxia, low pH, and deficiencies in nutrients. In order to survive harsh tumor microenvironments, cancer cells actively utilize adaptive stress pathways such as the integrated stress response (ISR) (Ye et al., “The GCN2-ATF4 pathway is critical for tumor cell survival and proliferation in response to nutrient deprivation,”, EMBO J., 2010, 29(12): 2082-2096; Pakos-Zebrucka et al., “The integrated stress response,” EMBO Rep., 2016, 17(10): 1374-1395). The ISR consists of 4 kinases; protein kinase ribonucleic acid (RNA)-like endoplasmic reticulum kinase, protein kinase double-stranded RNA-dependent, general control nondepressible 2 (GCN2), and heme-regulated inhibitor (Donnelly et al, “The eIF2α kinases: their structures and functions,” Cell Mol Life Sci., 2013, 70(19): 3493-3511). These four kinases sense unique stressors and phosphorylate the α-subunit of eukaryotic initiation factor 2 (eIF2α) (Albert et al., “Adaptive Protein Translation by the Integrated Stress Response Maintains the Proliferative and Migratory Capacity of Lung Adenocarcinoma Cells,” Mol Cancer Res, 2019, 17(12): 2343-2355). The high molecular weight kinase GCN2 senses amino acid deficiency as part of the ISR. Under amino acid starvation, uncharged transfer RNA accumulates and activates GCN2 (Anda et al., “Activation of Gen2 in response to different stresses,” PLOS ONE, 2017, 12(8): E0182143). Phosphorylation of eIF2α by ISR kinases, such as GCN2, inhibits general protein synthesis during cellular stress but also promotes the translation of select mRNAs including activating transcription factor 4 (ATF4), which is a key effector of the ISR (Pakos-Zebrucka et al.). Once translated, ATF4 translocates to the nucleus and drives the expression of genes involved in adaptation to stress such as autophagy, antioxidant response, amino acid biosynthesis, and metabolism (Pakos-Zebrucka et al.; Harding et al., “An integrated stress response regulates amino acid metabolism and resistance to oxidative stress,” Mol Cell, 2003, 11(3): 619-633). Other factors which activate GCN2 include ultraviolet light, viral infection, and oxidative stress (Costa-Mattioli et al., “The integrated stress response: From mechanism to disease,” Science, 2020, 368(6489): eaat5314). ATF4 is important for tumor cells to maintain homeostasis of amino acid metabolism. Activation of the ISR pathway promotes tumor cell survival under nutrient deprivation (Ye et al.). GCN2/ATF4 expression is elevated in primary human liver, breast, lung, and head and neck tumors, and GCN2 activation compared to normal tissue has been observed in colon, breast, and lung tumor samples.

ISR activation plays a dual role in cell fate decisions. During acute stress conditions, ISR can promote adaptation; during chronic stress conditions, this pathway can become apoptotic, which results in increased phosphorylation of eIF2α for an extended time (Wortel et al., “Surviving Stress: Modulation of ATF4-Mediated Stress Responses in Normal and Malignant Cells,” Trends Endocrinol Metabol., 2017, 28(11): 794-806). By reducing protein synthesis or activating apoptotic pathways, prolonged activation of ISR can be harmful to cell growth (Wortel et al.; Harding et al., “Ppplr14 gene knockout reveals an essential role for translation initiation factor 2 alpha (eIF2alpha) dephosphorylation in mammalian development,” Proc Natl Acad Sci USA, 2009, 106(6); 1832-1837; Munch, “The different axes of the mammalian mitochondrial unfolded protein response,” BMC Biology, 2018; 16(1): 81). Persistent ISR activation as a consequence of mutation of eIF2α phosphatases has been shown to have a deleterious effect on embryogenesis due to inhibition of protein synthesis (Harding et al., “Ppplr14 gene knockout reveals an essential role for translation initiation factor 2 alpha (eIF2alpha) dephosphorylation in mammalian development,” Proc Natl Acad Sci USA, 2009, 106(6); 1832-1837). GCN2 activation also can have antiproliferative effects through suppression of general protein synthesis and induction of cell cycle arrest preventing cells from growing during times of nutrient scarcity (Lehman et al, “Translation Upregulation of an Individual p21Cip1 Transcript Variant by GCN2 Regulates Cell Proliferation and Survival under Nutrient Stress,” PLOS Genetics, 2015, 11(6): e1005212). Therefore, continuous activation of the GCN2 pathway could suppress protein synthesis and cell growth, thereby inhibiting tumor proliferation.

Thus, there remains an unmet need to develop new therapeutic strategies that utilize modulation (e.g., activation or inhibition) of the GCN2 pathway for the treatment of a variety of cancers (e.g., advanced solid tumors and blood cancers).

### SUMMARY

In one aspect, provided herein is a method of inhibiting myeloid-derived suppressor cells (MDSCs) in a subject in need thereof, comprising administering to the subject a therapeutically effective amount of a compound of formula (I)

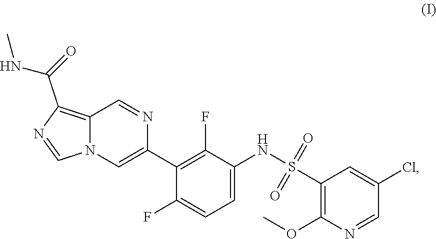 [SMILES: CNC(=O)c1ncn2cc(-c3c(F)ccc(NS(=O)(=O)c4cc(Cl)cnc4OC)c3F)ncc12]

or a pharmaceutically acceptable salt thereof. In certain embodiments, the method activates the integrated stress response (ISR) in the MDSCs.

In another aspect, provided herein is a method of treating metastases in a subject in need thereof, comprising administering to the subject a therapeutically effective amount of a compound of formula (I)

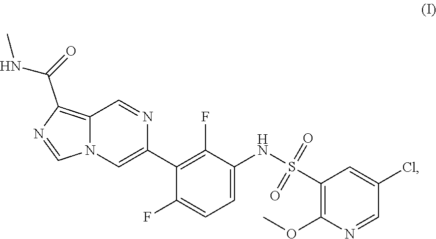 [SMILES: CNC(=O)c1ncn2cc(-c3c(F)ccc(NS(=O)(=O)c4cc(Cl)cnc4OC)c3F)ncc12]

or a pharmaceutically acceptable salt thereof.

In another aspect, provided herein is a method of reducing tumor volume of metastases in a subject in need thereof, comprising administering to the subject a therapeutically effective amount of a compound of formula (I)

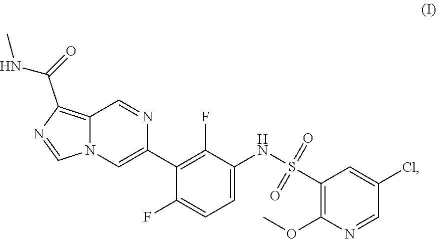 [SMILES: CNC(=O)c1ncn2cc(-c3c(F)ccc(NS(=O)(=O)c4cc(Cl)cnc4OC)c3F)ncc12]

or a pharmaceutically acceptable salt thereof.

In certain embodiments, the metastases are present in one or more sites selected from the group consisting of the subject's lung, liver, bone, brain, peritoneum, adrenal gland, skin, and muscle. In certain embodiments, the metastases have metastasized from a primary tumor. In certain embodiments, the primary tumor is a solid tumor. In certain embodiments, the primary tumor is selected from the group consisting of a breast cancer, non-small cell lung cancer (NSCLC), head and neck cancer, colorectal cancer, ovarian cancer, and hepatocellular carcinoma. In certain embodiments, the primary tumor is a breast cancer.

In certain embodiments, the compound of formula (I), or a pharmaceutically acceptable salt thereof, is administered once daily. In certain embodiments, the compound of formula (I), or a pharmaceutically acceptable salt thereof, is administered in an amount of about 10 mg to about 75 mg once daily. In certain embodiments, the compound of formula (I), or a pharmaceutically acceptable salt thereof, is administered in an amount of about 75 mg to about 150 mg once daily. In certain embodiments, the compound of formula (I), or a pharmaceutically acceptable salt thereof, is administered over a period of 2 years.

In certain embodiments, the compound of formula (I), or pharmaceutically acceptable salt thereof, is administered orally. In certain embodiments, the compound of formula (I), or a pharmaceutically acceptable salt thereof, is administered in the form of a capsule. In certain embodiments, the method reduces the inhibition of T cells in the subject relative to a subject receiving placebo. In certain embodiments, the method increases the activation of the ISR in MDSCs in the subject relative to a subject receiving placebo.

In certain embodiments, the method decreases the frequency of MDSCs in the lungs, spleen, and/or blood of the subject relative to a subject receiving placebo. In certain embodiments, the method increases MDSC expression of CD86 and/or MHCII in the subject relative to a subject receiving placebo. In certain embodiments, the method reduces S100 levels in CD11b+ cells in the subject relative to a subject receiving placebo. In certain embodiments, the method reduces S100 levels in peripheral blood mononuclear cells (PBMCs) and/or plasma in the subject relative to a subject receiving placebo. In certain embodiments, the method increases L-2, Ki67, and/or T-bet on CD4 T-cells in the subject relative to a subject receiving placebo. In certain embodiments, the method increases IL-2, Ki67, and/or T-bet on CD8 T-cells in the subject relative to a subject receiving placebo. In certain embodiments, the method increases TSR pathway activation in the subject in MDSCs to a greater extent than in T-cells.

In certain embodiments, the compound of formula (I), or a pharmaceutically acceptable salt thereof, is administered as a pharmaceutically acceptable salt of the compound of formula (I). In certain embodiments, the pharmaceutically acceptable salt is a potassium salt. In certain embodiments, the potassium salt is a potassium salt hydrate. In certain embodiments, the potassium salt hydrate is a potassium salt monohydrate.

### BRIEF DESCRIPTION OF THE FIGURES

FIGS. 1A-1B show effects of HC-7366 on metastases of 4T1 tumor cells within the lungs of allografted mice. FIG. 1A shows images of hematoxylin and eosin-stained lungs of allografted mice treated with indicated doses of vehicle or HC-7366. FIG. 1B is a graph showing quantification of metastases within imaged tissue sections of allografted mice treated with indicated doses of vehicle or HC-7366.

FIG. 2 is a graph showing number of lung metastases in 4T1 allografted mice treated with indicated dosages of vehicle, HC-7366, and/or anti-PD-1 therapy.

FIG. 3 is a timeline of an experiment testing the effects of HC-7366 on metastases in a 4T1 orthotopic allograft model.

FIG. 4 shows graphs of tumor volumes overtime of allografted mice treated with indicated doses of vehicle or HC-7366 in three separate experiments

FIGS. 5A-5C show results of experiments measuring metastases in 4T1 allografted mice treated with vehicle, 3 mg/kg HC-7366, or 30 mg/kg HC-7366. FIG. 5A shows percentage of metastatic area in lung samples collected from 4T1 allografted mice. FIG. 5B shows the gross count of metastases in lung samples collected from 4T1 allografted mice. FIG. 5C shows percentage of metastatic area in lung samples collected from 4T1 allografted mice.

FIGS. 6A-6C are hematoxylin and eosin stained lung samples collected from 4T1 allografted mice treated with vehicle (FIG. 6A), 3 mg/kg HC-7366 (FIG. 6B), or 30 mg/kg HC-7366 (FIG. 6C).

FIGS. 7A-7C show IHC staining of 4T1 allografted mice treated with vehicle or 3 mg/kg HC-7366. FIG. 7A shows images of lung samples from 4T1-allografted mice. FIG. 7B shows quantification of ASNS (left panel) and PSAT1 (right panel) in images of tissues taken from allografted mice treated with DMSO or indicated dosages of HC-7366. FIG. 7C shows normalized protein levels of ISR markers PSAT (top left panel), ATF4 (top right panel), TRIB3 (bottom left panel), and ASNS (bottom right panel).

FIGS. 8A-8B show effects of HC-7366 treatment on the spleens of 4T1 allografted mice. FIG. 8A shows flow cytometry analysis of cells collected from the spleens of 4T1 allografted mice. FIG. 8B shows spleen weights of mice in three separate experiments.

FIG. 9 shows gene expression analysis of myeloid cells isolated from tumors (left panel) or spleen (right panel) of 4T1 allografted mice treated with vehicle or indicated dosages of HC-7366. Rows=individual mice. 77 genes surveyed, differentially expressed genes are shown.

FIGS. 10A-10C show quantification of myeloid-derived suppressor cells (MDSCs) in lungs and spleen of 4T1 allografted mice treated with vehicle or indicated dosages of HC-7366. FIG. 10A shows number of MDSCs in lungs (left panel) or spleen (right panel) of allografted mice treated with vehicle or indicated dosages of HC-7366 at indicated time points post-tumor engraftment. FIG. 10B shows quantification of CD86 (left panel) or MHCII (right panel) staining on MDSCs collected from the lungs of allografted mice treated with vehicle or indicated dosages of HC-7366 at day 14 post-tumor engraftment. FIG. 10C shows quantification of CD86 (left panel) or MHCII (right panel) staining on MDSCs collected from the spleen of allografted mice treated with vehicle or indicated dosages of HC-7366 at day 14 post-tumor engraftment.

FIGS. 11A-11B shows an experiment detailing ex vivo treatment of bone marrow-derived MDSCs from 4T1 allografted mice. FIG. 11A shows a schematic of the isolation and purification of MDSCs and T cells from 4T1 allografted mice. FIG. 11B shows proliferation of CD4 T cells (left panel) and CD8 T cells (right panel) following incubation with isolated MDSCs, tryptophan-containing medium, and HC-7366 as indicated.

FIGS. 12A-12F show staining of lung samples from 4T1 allografted mice treated with vehicle or indicated dosages of HC-7366. FIG. 12A shows images of lung samples stained for CD4 from allografted mice treated with vehicle or indicated dosages of HC-7366. FIG. 12B shows images of lung samples stained for CD8 from allografted mice treated with vehicle or indicated dosages of HC-7366. FIG. 12C shows quantification of percentages of CD8+(left panel) or CD4+(right panel) cells in lung metastases collected from 4T1 allografted mice. FIG. 12D shows quantification of percentages of CD8+(left panel) or CD4+(right panel) cells in normal lung tissue collected from 4T1 allografted mice FIG. 12E shows quantification of percentages of CD8+/Ki67+(left panel) or CD4+/Ki67+(right panel) cells in lung metastases collected from 4T1 allografted mice. FIG. 12F shows quantification of percentages of CD8+/Ki67+(left panel) or CD4+/Ki67+(right panel) cells in normal lung tissue collected from 4T1 allografted mice.

FIGS. 13A-13D show graphs of quantification of proliferation and activation markers of CD4 T cells collected from the lungs or spleen of 4T1 allografted mice treated with vehicle or indicated dosages of HC-7366 at 21 days post-tumor engraftment. FIG. 13A shows graphs quantifying staining of IL-2 (left panel), Ki-67 (center panel), or granzyme B (right panel) in CD8 T cells collected from the lungs of 4T1 allografted mice treated with vehicle or indicated dosages of HC-7366 after 21 days of treatment. FIG. 13B shows graphs quantifying staining of IL-2 (left panel), Ki-67 (center panel), or T-bet (right panel) in CD4 T cells collected from the lungs of 4T1 allografted mice treated with vehicle or indicated dosages of HC-7366 after 21 days post-tumor engraftment. FIG. 13C shows graphs quantifying staining of IL-2 (left panel), Ki-67 (center panel), or granzyme B (right panel) in CD8 T cells collected from the spleens of 4T1 allografted mice treated with vehicle or indicated dosages of HC-7366 after 21 days of treatment. FIG. 13D shows graphs quantifying staining of IL-2 (left panel), Ki-67 (center panel), or T-bet (right panel) in CD4 T cells collected from the spleens of 4T1 allografted mice treated with vehicle or indicated dosages of HC-7366 at 21 days post-tumor engraftment.

FIGS. 14A-14B show CD98 staining of cells collected from 4T1 allografted mice. FIG. 14A shows graphs showing CD98 staining of MDSCs (left panel) or CD8 T cells (right panel) collected from the lungs of 4T1 allografted mice treated with vehicle or indicated dosages of HC-7366 FIG. 14B shows graphs showing CD98 staining of MDSCs (left panel) or CD8 T cells (right panel) collected from the spleens of 4T1 allografted mice treated with vehicle or indicated dosages of HC-7366.

FIGS. 15A-15C show analysis of lung metastases in lung samples from 4T1 allografted mice treated with vehicle or indicated dosages of HC-7366. FIG. 15A shows images of metastases (top row) or normal lung tissue (bottom row) stained for S100A8, S100A9, and CD11b. FIG. 15B shows graphs quantifying percentage of CD11b+ cells that stain positive for S100A8/9 in metastatic tissue (left panel) or normal lung tissue (right panel). FIG. 15C shows normalized protein expression of S100A9 in PBMCs (left panel) or concentration of S100A8/9 in plasma (right panel).

### DETAILED DESCRIPTION

As generally described herein, the present disclosure provides methods of inhibiting myeloid-derived suppressor cells (MDSCs) in a subject in need thereof. The present disclosure also provides methods of treating or reducing the tumor volumes of metastases in a subject in need thereof. The methods described herein generally comprise administering to the subject an effective amount of the compound of formula (I), or a pharmaceutically acceptable salt thereof.

### Definitions

To facilitate an understanding of the present invention, a number of terms and phrases are defined below.

Unless defined otherwise, all technical and scientific terms used herein have the same meaning as commonly understood by one of ordinary skill in the art to which this invention belongs. The abbreviations used herein have their conventional meaning within the chemical and biological arts. The chemical structures and formulae set forth herein are constructed according to the standard rules of chemical valency known in the chemical arts.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The articles “a” and “an” are used in this disclosure to refer to one or more than one (i.e., to at least one) of the grammatical object of the article, unless the context is inappropriate. By way of example, “an element” means one element or more than one element.

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred from the context.

At various places in the present specification, variables or parameters are disclosed in groups or in ranges. It is specifically intended that the description include each and every individual subcombination of the members of such groups and ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

As a general matter, compositions specifying a percentage are by weight unless otherwise specified. Further, if a variable is not accompanied by a definition, then the previous definition of the variable controls.

As used herein, “pharmaceutical composition” or “pharmaceutical formulation” refers to the combination of an active agent with a carrier, inert or active, making the composition especially suitable for diagnostic or therapeutic use in vivo or ex vivo.

“Pharmaceutically acceptable” means approved or approvable by a regulatory agency of the federal or a state government or the corresponding agency in countries other than the United States, or that is listed in the U.S. Pharmacopoeia or other generally recognized pharmacopoeia for use in animals, and more particularly, in humans.

As used herein, “pharmaceutically acceptable salt” refers to any salt of an acidic or a basic group that may be present in a compound of the present invention (e.g., a compound of formula (I)), which salt is compatible with pharmaceutical administration.

As is known to those of skill in the art, “salts” of compounds may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic and benzenesulfonic acid. Other acids, such as oxalic, while not in themselves pharmaceutically acceptable, may be employed in the preparation of salts useful as intermediates in obtaining the compounds described herein and their pharmaceutically acceptable acid addition salts.

Examples of bases include, but are not limited to, alkali metal (e.g., sodium and potassium) hydroxides, alkaline earth metal (e.g., magnesium and calcium) hydroxides, ammonia, and compounds of formula NW⁴⁺, wherein W is C₁₋₄ alkyl, and the like.

Examples of salts include, but are not limited to, acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, K⁺, Ca²⁺, NH⁺, and NW⁴⁺ (where W can be a C₁₋₄ alkyl group), and the like.

For therapeutic use, salts of the compounds of the present invention (e.g., a compound of formula (I)) are contemplated as being pharmaceutically acceptable. However, salts of acids and bases that are non-pharmaceutically acceptable may also find use, for example, in the preparation or purification of a pharmaceutically acceptable compound.

As used herein, “pharmaceutically acceptable excipient” refers to a substance that aids the administration of an active agent to and/or absorption by a subject and can be included in the compositions of the present invention without causing a significant adverse toxicological effect on the patient. Non-limiting examples of pharmaceutically acceptable excipients include water, NaCl, normal saline solutions, such as a phosphate buffered saline solution, emulsions (e.g., such as an oil/water or water/oil emulsions), lactated Ringer's, normal sucrose, normal glucose, binders, fillers, disintegrants, lubricants, coatings, sweeteners, flavors, salt solutions (such as Ringer's solution), alcohols, oils, gelatins, carbohydrates, fatty acid esters, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. For examples of excipients, see Martin, Remington's Pharmaceutical Sciences, 15th Ed., Mack Publ. Co., Easton, PA (1975).

The term “AUC” refers to the area under the time/plasma concentration curve after administration of the compound of formula (I), or pharmaceutically acceptable salt thereof, or a pharmaceutical composition described herein. AUC_{0-infinity} denotes the area under the plasma concentration versus time curve from time 0 to infinity AUC₀₋ₜ denotes the area under the plasma concentration versus time curve from time 0 to time t. It should be appreciated that AUC values can be determined by known methods in the art.

A “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or non-human animals, e.g., mammals such as primates (e.g., cynomolgus monkeys, rhesus monkeys), cattle, pigs, horses, sheep, goats, rodents, cats, and/or dogs. In certain embodiments, the subject is a human. In certain embodiments, the subject is an adult human. In certain embodiments, the subject is a non-human animal.

The term “Cₘₐₓ” refers to the maximum concentration of a therapeutic agent in the blood (e.g., plasma) following administration of the therapeutic agent (e.g., the compound of formula (I), or a pharmaceutically acceptable salt thereof) or a pharmaceutical composition comprising the therapeutic agent (e.g., a pharmaceutical composition described herein).

The term “tₘₐₓ” refers to the time in hours when Cₘₐₓ is achieved following administration of a therapeutic agent (e.g., a compound of formula (I), or a pharmaceutically acceptable salt thereof) or a pharmaceutical composition comprising the therapeutic agent (e.g., a pharmaceutical composition described herein).

As used herein, “solid dosage form” means a pharmaceutical dose(s) in solid form, e.g., tablets, capsules, granules, powders, sachets, reconstitutable powders, dry powder inhalers and chewables.

As used herein, “administering” means oral administration, administration as a suppository, topical contact, intravenous administration, parenteral administration, intraperitoneal administration, intramuscular administration, intralesional administration, intrathecal administration, intracranial administration, intranasal administration or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arterial, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g., anti-cancer agent, chemotherapeutic, or immunotherapy). The compound of formula (I), or a pharmaceutically acceptable salt thereof, can be administered alone or can be co-administered to the patient Co-administration is meant to include simultaneous or sequential administration of the compound individually or in combination (more than one compound or agent). Thus, the preparations can also be combined, when desired, with other active substances (e.g., to reduce metabolic degradation).

As used herein, “fasting state” means at least 1 hour before food or at least 2 hours after food is consumed by a subject.

The terms “disease,” “disorder,” and “condition” are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (e.g., “therapeutic treatment”).

In general, an “effective amount” or a “therapeutically effective amount” of a compound (e.g., a compound of formula (I), or a pharmaceutically acceptable salt thereof) refers to an amount sufficient to elicit the desired biological response, e.g., to treat an advanced solid tumor and/or a blood cancer. As will be appreciated by those of ordinary skill in this art, the effective amount of a compound of the disclosure may vary depending on such factors as the desired biological endpoint, the pharmacokinetics of the compound, the disease being treated, the mode of administration, and the age, weight, health, and condition of the subject.

### Compound

The compound of formula (I), as depicted below, is a selective GCN2 modulator (e.g., activation or inhibition of GCN2), and is also known as 6-(3-((5-chloro-2-methoxypyridine)-3-sulfonamido)-2,6-difluorophenyl)-N-methylimidazo[1,5-a]pyrazine-1-carboxamide.

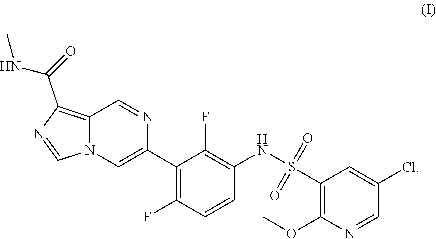 [SMILES: CNC(=O)c1ncn2cc(-c3c(F)ccc(NS(=O)(=O)c4cc(Cl)cnc4OC)c3F)ncc12]

The compound of formula (I) is also referred to as HC-7366, HC-7366K, or HC-GCN2m throughout the present disclosure. A method of chemically synthesizing the compound of formula (I) is described in Example 1.

In one aspect, provided herein is a method of administering the compound of formula (I), or a pharmaceutically acceptable salt thereof, for the inhibition of myeloid-derived suppressor cells (MDSCs) in a subject in need thereof.

In another aspect, provided herein is a method of administering the compound of formula (I), or a pharmaceutically acceptable salt thereof, for the treatment of metastases in a subject in need thereof.

In various embodiments, provided herein is a method of administering a pharmaceutically acceptable salt of the compound of formula (I) for the reduction of tumor volume of metastases in a subject in need thereof.

In certain embodiments, the pharmaceutically acceptable salt of the compound of formula (I) is a potassium salt. In certain embodiments, the potassium salt of the compound of formula (I) is a hydrate. In certain embodiments, the potassium salt of the compound of formula (I) is a monohydrate.

### Pharmaceutical Compositions

Provided herein are pharmaceutical compositions generally comprising a compound of formula (I), or a pharmaceutically acceptable salt thereof, and one or more pharmaceutically acceptable excipients.

In one aspect, provided herein is a method of administering a pharmaceutical composition comprising a compound of formula (I), or a pharmaceutically acceptable salt thereof, and one or more pharmaceutically excipients, for inhibition of myeloid-derived suppressor cells (MDSCs) in a subject in need thereof.

In another aspect, provided herein is a method of administering a pharmaceutical composition comprising a compound of formula (I), or a pharmaceutically acceptable salt thereof, and one or more pharmaceutically excipients, for the treatment of metastases in a subject in need thereof.

In another aspect, provided herein is a method of administering a pharmaceutical composition comprising a pharmaceutically acceptable salt of a compound of formula (I) and one or more pharmaceutically excipients, for the reduction of tumor volume of metastases in a subject in need thereof.

In various embodiments, provided herein is a pharmaceutical composition comprising an effective amount of a compound of formula (I), or a pharmaceutically acceptable salt thereof, and one or more pharmaceutically acceptable excipients.

In various embodiments, provided herein is a pharmaceutical composition comprising an effective amount of a pharmaceutically acceptable salt of a compound of formula (I) and one or more pharmaceutically acceptable excipients.

In certain embodiments, the amount of the compound of formula (I), or a pharmaceutically acceptable salt thereof, in a pharmaceutical composition described herein is about 10 mg to about 150 mg, about 20 mg to about 150 mg, about 40 mg to about 150 mg, about 75 mg to about 150 mg, about 125 mg to about 150 mg, about 10 mg to about 125 mg, about 10 mg to about 75 mg, about 10 mg to about 40 mg, about 10 mg to about 20 mg, about 20 mg to about 125 mg, about 20 mg to about 75 mg, about 20 mg to about 40 mg, about 40 mg to about 125 mg, about 40 mg to about 75 mg, or about 75 mg to about 125 mg, on a free acid equivalent weight basis. In certain embodiments, the amount of the compound of formula (I), or a pharmaceutically acceptable salt thereof, in a pharmaceutical composition described herein is about 10 mg to about 150 mg on a free acid equivalent weight basis.

In certain embodiments, the amount of the compound of formula (I), or a pharmaceutically acceptable salt thereof, in a pharmaceutical composition described herein is about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 125 mg, or about 150 mg, on a free acid equivalent weight basis. In certain embodiments, the amount of the compound of formula (I), or a pharmaceutically acceptable salt thereof, in a pharmaceutical composition described herein is about 10 mg on a free acid equivalent weight basis. In certain embodiments, the amount of the compound of formula (I), or a pharmaceutically acceptable salt thereof, in a pharmaceutical composition described herein is about 20 mg on a free acid equivalent weight basis. In certain embodiments, the amount of the compound of formula (I), or a pharmaceutically acceptable salt thereof, in a pharmaceutical composition described herein is about 40 mg on a free acid equivalent weight basis. In certain embodiments, the amount of the compound of formula (I), or a pharmaceutically acceptable salt thereof, in a pharmaceutical composition described herein is about 75 mg on a free acid equivalent weight basis. In certain embodiments, the amount of the compound of formula (I), or a pharmaceutically acceptable salt thereof, in a pharmaceutical composition described herein is about 125 mg on a free acid equivalent weight basis. In certain embodiments, the amount of the compound of formula (I), or a pharmaceutically acceptable salt thereof, in a pharmaceutical composition described herein is about 150 mg on a free acid equivalent weight basis.

In certain embodiments, the amount of the pharmaceutically acceptable salt of the compound of formula (I) in a pharmaceutical composition described herein is about 10 mg to about 150 mg, about 20 mg to about 150 mg, about 40 mg to about 150 mg, about 75 mg to about 150 mg, about 125 mg to about 150 mg, about 10 mg to about 125 mg, about 10 mg to about 75 mg, about 10 mg to about 40 mg, about 10 mg to about 20 mg, about 20 mg to about 125 mg, about 20 mg to about 75 mg, about 20 mg to about 40 mg, about 40 mg to about 125 mg, about 40 mg to about 75 mg, or about 75 mg to about 125 mg, on a free acid equivalent weight basis. In certain embodiments, the amount of the pharmaceutically acceptable salt of the compound of formula (I) in a pharmaceutical composition described herein is about 10 mg to about 150 mg on a free acid equivalent weight basis.

In certain embodiments, the amount of the pharmaceutically acceptable salt of the compound of formula (I) in a pharmaceutical composition described herein is about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 125 mg, or about 150 mg, on a free acid equivalent weight basis. In certain embodiments, the amount of the pharmaceutically acceptable salt of the compound of formula (I) in a pharmaceutical composition described herein is about 10 mg on a free acid equivalent weight basis. In certain embodiments, the amount of the pharmaceutically acceptable salt of the compound of formula (I) in a pharmaceutical composition described herein is about 20 mg on a free acid equivalent weight basis. In certain embodiments, the amount of the pharmaceutically acceptable salt of the compound of formula (I) in a pharmaceutical composition described herein is about 40 mg on a free acid equivalent weight basis. In certain embodiments, the amount of the pharmaceutically acceptable salt of the compound of formula (I) in a pharmaceutical composition described herein is about 75 mg on a free acid equivalent weight basis. In certain embodiments, the amount of the pharmaceutically acceptable salt of the compound of formula (I) in a pharmaceutical composition described herein is about 125 mg on a free acid equivalent weight basis. In certain embodiments, the amount of the pharmaceutically acceptable salt of the compound of formula (I) in a pharmaceutical composition described herein is about 150 mg on a free acid equivalent weight basis.

Tn various embodiments, provided herein are pharmaceutical compositions comprising: (i) about 10 mg to about 150 mg of a compound of formula (I), or a pharmaceutically acceptable salt thereof, on a free acid equivalent weight basis; and (ii) one or more pharmaceutically acceptable excipients.

In various embodiments, provided herein are pharmaceutical compositions comprising: (i) about 10 mg to about 150 mg of a pharmaceutically acceptable salt of the compound of formula (I), on a free acid equivalent weight basis; and (ii) one or more pharmaceutically acceptable excipients.

In another aspect, provided herein are pharmaceutical compositions comprising about 10 mg to about 150 mg of a compound of formula (I), or a pharmaceutically acceptable salt thereof, on a free acid equivalent weight basis, and one or more pharmaceutically acceptable excipients, for the inhibition of myeloid-derived suppressor cells (MDSCs) in a subject in need thereof.

In another aspect, provided herein are pharmaceutical compositions comprising about 10 mg to about 150 mg of a compound of formula (I), or a pharmaceutically acceptable salt thereof, on a free acid equivalent weight basis, and one or more pharmaceutically acceptable excipients, for the treatment of metastases in a subject in need thereof.

In another aspect, provided herein are pharmaceutical compositions comprising about 10 mg to about 150 mg of a compound of formula (I), or a pharmaceutically acceptable salt thereof, on a free acid equivalent weight basis, and one or more pharmaceutically acceptable excipients, for the reduction of tumor volume of metastases in a subject in need thereof.

In another aspect, provided herein are pharmaceutical compositions comprising about 10 mg to about 150 mg of a pharmaceutically acceptable salt of the compound of formula (I), on a free acid equivalent weight basis, and one or more pharmaceutically acceptable excipients, for the inhibition of myeloid-derived suppressor cells (MDSCs) in a subject in need thereof.

In another aspect, provided herein are pharmaceutical compositions comprising about 10 mg to about 150 mg of a pharmaceutically acceptable salt of the compound of formula (I), on a free acid equivalent weight basis, and one or more pharmaceutically acceptable excipients, for the treatment of metastases in a subject in need thereof.

In another aspect, provided herein are pharmaceutical compositions comprising about 10 mg to about 150 mg of a pharmaceutically acceptable salt of the compound of formula (I), on a free acid equivalent weight basis, and one or more pharmaceutically acceptable excipients, for the reduction of tumor volume of metastases in a subject in need thereof.

In certain embodiments, the pharmaceutically acceptable salt of the compound of formula (I) is a potassium salt. In certain embodiments, the potassium salt of the compound of formula (I) is a hydrate. In certain embodiments, the potassium salt of the compound of formula (I) is a monohydrate.

The pharmaceutical compositions described herein can be administered by a variety of routes including, but not limited to, oral (enteral) administration, parenteral (by injection) administration, rectal administration, transdermal administration, intradermal administration, intrathecal administration, subcutaneous (SC) administration, intravenous (TV) administration, intramuscular (IM) administration, and intranasal administration. In certain embodiments, the pharmaceutical compositions described herein are administered orally.

The pharmaceutical compositions described herein may also be administered chronically (“chronic administration”). Chronic administration refers to administration of a compound or pharmaceutical composition thereof over an extended period of time, e.g., for example, over 3 months, 6 months, 1 year, 2 years, 3 years, 5 years, etc., or may be continued indefinitely, for example, for the rest of the subject's life. In certain embodiments, the chronic administration is intended to provide a constant level of the compound in the blood, e.g., within the therapeutic window over the extended period of time.

The pharmaceutical compositions described herein may be presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions.

In certain embodiments, the pharmaceutical compositions provided herein are administered to the patient as a solid dosage form. In certain embodiments, the solid dosage form is a capsule.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation. General considerations in the formulation and/or manufacture of pharmaceutical compositions can be found, for example, in Remington: The Science and Practice of Pharmacy 21^{st} ed., Lippincott Williams & Wilkins, 2005.

### Methods of Use and Treatment

Provided herein are methods of inhibiting myeloid-derived suppressor cells (MDSCs) in a subject in need thereof. The methods generally comprise administering to the subject an effective amount of a compound of formula (I), or a pharmaceutically acceptable salt thereof; or a pharmaceutical composition described herein.

Myeloid-derived suppressor cells (MDSCs) are immature myeloid cells known to inhibit anti-tumor T cell immunity. MDSCs can promote tumor metastasis by promoting extracellular matrix (ECM) remodeling, enhancing extravasation of circulating tumor cells (CTCs) at metastatic sites, and promoting secretion of pro-angiogenic factors VEGFA and MMP9. MDSCs can also indirectly promote tumor metastasis by enhancing immunosuppression in the tumor environment by depletion of essential metabolites, production of reactive oxygen species (ROS) and reactive nitrogen species (RNS), contact-mediated inhibition of immune cells, release of soluble inhibitory factors, and generation of other immunosuppressive cells (e.g., T_{reg} cells). The abundance of these cells in the tumor environment correlates negatively with cancer patient survival. Higher degree of ISR activation is known to mark primitive myeloid cells, so hyperactivation of the ISR in MDSCs can cause cytotoxicity of MDSCs and thus serve to inhibit tumor growth, angiogenesis, and metastasis.

In certain embodiments, the method increases the activation of the ISR in MDSCs in the subject relative to a subject receiving placebo. In certain embodiments, the method decreases the frequency of MDSCs in the lungs, spleen, and/or blood of the subject relative to a subject receiving placebo. In certain embodiments, the method increases MDSC expression of CD86 and/or MHCII in the subject relative to a subject receiving placebo. In certain embodiments, the method reduces S100 levels in CD11b+ cells in the subject relative to a subject receiving placebo.

In certain embodiments, the method reduces S100 levels in peripheral blood mononuclear cells (PBMCs) and/or plasma in the subject relative to a subject receiving placebo. In certain embodiments, the method increases IL-2, Ki67, and/or T-bet on CD4 T-cells in the subject relative to a subject receiving placebo. In certain embodiments, the method increases IL-2, Ki67, and/or T-bet on CD8 T-cells in the subject relative to a subject receiving placebo. In certain embodiments, the method increases ISR pathway activation in the subject in MDSCs to a greater extent than in T-cells.

Also provided herein are methods of treating a solid tumor in a subject in need thereof. The methods generally comprise administering to the subject an effective amount of a compound of formula (I), or a pharmaceutically acceptable salt thereof; or a pharmaceutical composition described herein. In certain embodiments, the solid tumor has high degrees of MDSC infiltration. In certain embodiments, the solid tumor is an advanced solid tumor. In certain embodiments, the solid tumor is a metastatic tumor. In certain embodiments, the solid tumor is a secondary tumor.

Solid tumors the compound of formula (I), or a pharmaceutically acceptable salt thereof, are contemplated to be useful in treating include, but are not limited to, pancreatic cancer; bladder cancer, colorectal cancer, breast cancer, including metastatic breast cancer, prostate cancer, including androgen-dependent and androgen-independent prostate cancer; kidney or renal cancer, including, e.g., metastatic renal cell carcinoma; hepatocellular cancer, lung cancer, including, e.g., NSCLC, bronchioloalveolar carcinoma (BAC), and adenocarcinoma of the lung; ovarian cancer, including, e.g., progressive epithelial or primary peritoneal cancer; cervical cancer; gastric cancer; esophageal cancer, head and neck cancer, including, e.g., squamous cell carcinoma of the head and neck; melanoma; neuroendocrine cancer, including metastatic neuroendocrine tumors; brain tumors, including, e.g., glioma, anaplastic oligodendroglioma, adult glioblastoma multiforme, and adult anaplastic astrocytoma; bone cancer, and soft tissue sarcoma, hepatic carcinoma, rectal cancer, penile carcinoma, vulval cancer, thyroid cancer, salivary gland carcinoma, endometrial or uterine carcinoma, hepatoma, hepatocellular cancer, liver cancer, gastric or stomach cancer including gastrointestinal cancer, cancer of the peritoneum, squamous carcinoma of the lung, gastroesophageal cancer, biliary tract cancer, gall bladder cancer, colorectal/appendiceal cancer, and squamous cell cancer (e.g., epithelial squamous cell cancer).

In certain embodiments, the advanced solid tumor is selected from the group consisting of breast cancer, NSCLC, head and neck cancer, colorectal cancer, ovarian cancer, and hepatocellular carcinoma.

In certain embodiments, the metastases are present in one or more sites selected from the group consisting of the subject's lung, liver, bone, brain, peritoneum, adrenal gland, skin, and muscle.

In certain embodiments, administering an effective amount of the compound of formula (I), or a pharmaceutically acceptable salt thereof, comprises administering to the subject about 10 mg to about 150 mg, about 15 mg to about 150 mg, about 20 mg to about 150 mg, about 25 mg to about 150 mg, about 30 mg to about 150 mg, about 35 mg to about 150 mg, about 40 mg to about 150 mg, about 45 mg to about 150 mg, about 50 mg to about 150 mg, about 55 mg to about 150 mg, about 60 mg to about 150 mg, about 65 mg to about 150 mg, about 70 mg to about 150 mg, about 75 mg to about 150 mg, about 80 mg to about 150 mg, about 85 mg to about 150 mg, about 90 mg to about 150 mg, about 95 mg to about 150 mg, about 100 mg to about 150 mg, about 105 mg to about 150 mg, about 110 mg to about 150 mg, about 115 mg to about 150 mg, about 120 mg to about 150 mg, about 125 mg to about 150 mg, about 130 mg to about 150 mg, about 135 mg to about 150 mg, about 140 mg to about 150 mg, or about 145 mg to about 150 mg of the compound of formula (I), or a pharmaceutically acceptable salt thereof, on a free acid equivalent weight basis.

In certain embodiments, administering an effective amount of the compound of formula (I), or a pharmaceutically acceptable salt thereof, comprises administering to the subject about 10 mg to about 150 mg, about 15 mg to about 145 mg, about 20 mg to about 140 mg, about 25 mg to about 135 mg, about 30 mg to about 135 mg, about 35 mg to about 130 mg, about 40 mg to about 125 mg, about 45 mg to about 120 mg, about 50 mg to about 115 mg, about 55 mg to about 110 mg, about 60 mg to about 105 mg, about 65 mg to about 100 mg, about 70 mg to about 95 mg, about 75 mg to about 90 mg, or about 80 mg to about 85 mg of the compound of formula (I), or a pharmaceutically acceptable salt thereof, on a free acid equivalent weight basis.

In certain embodiments, administering an effective amount of the compound of formula (I), or a pharmaceutically acceptable salt thereof, comprises administering to the subject about 10 mg to about 75 mg, about 15 mg to about 75 mg, about 20 mg to about 75 mg, about 25 mg to about 75 mg, about 30 mg to about 75 mg, about 35 mg to about 75 mg, about 40 mg to about 75 mg, about 45 mg to about 75 mg, about 50 mg to about 75, about 55 mg to about 75 mg, about 60 mg to about 75 mg, about 65 mg to about 75 mg, or about 70 mg to about 75 mg of the compound of formula (I), or a pharmaceutically acceptable salt thereof, on a free acid equivalent weight basis.

In certain embodiments, administering an effective amount of the compound of formula (I), or a pharmaceutically acceptable salt thereof, comprises administering to the subject about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, or about 150 mg of the compound of formula (I), or a pharmaceutically acceptable salt thereof, on a free acid equivalent weight basis.

In certain embodiments, administering an effective amount of the compound of formula (I), or a pharmaceutically acceptable salt thereof, comprises administering to the subject about 10 mg of the compound of formula (I), or a pharmaceutically acceptable salt thereof, on a free acid equivalent weight basis. In certain embodiments, administering an effective amount of the compound of formula (I), or a pharmaceutically acceptable salt thereof, comprises administering to the subject about 20 mg of the compound of formula (I), or a pharmaceutically acceptable salt thereof, on a free acid equivalent weight basis. In certain embodiments, administering an effective amount of the compound of formula (I), or a pharmaceutically acceptable salt thereof, comprises administering to the subject about 40 mg of the compound of formula (I), or a pharmaceutically acceptable salt thereof, on a free acid equivalent weight basis. In certain embodiments, administering an effective amount of the compound of formula (I), or a pharmaceutically acceptable salt thereof, comprises administering to the subject about 75 mg of the compound of formula (I), or a pharmaceutically acceptable salt thereof, on a free acid equivalent weight basis. In certain embodiments, administering an effective amount of the compound of formula (I), or a pharmaceutically acceptable salt thereof, comprises administering to the subject about 125 mg of the compound of formula (I), or a pharmaceutically acceptable salt thereof, on a free acid equivalent weight basis. In certain embodiments, administering an effective amount of the compound of formula (I), or a pharmaceutically acceptable salt thereof, comprises administering to the subject about 150 mg of the compound of formula (I), or a pharmaceutically acceptable salt thereof, on a free acid equivalent weight basis.

In certain embodiments, administering an effective amount of the compound of formula (I), or a pharmaceutically acceptable salt thereof, comprises administering to the subject at least about 10 mg, at least about 15 mg, at least about 20 mg, at least about 25 mg, at least about 30 mg, at least about 35 mg, at least about 40 mg, at least about 45 mg, at least about 50 mg, at least about 55 mg, at least about 60 mg, at least about 65 mg, at least about 70 mg, at least about 75 mg, at least about 80 mg, at least about 85 mg, at least about 90 mg, at least about 95 mg, at least about 100 mg, at least about 105 mg, at least about 110 mg, at least about 115 mg, at least about 120 mg, at least about 125 mg, at least about 130 mg, at least about 135 mg, at least about 140 mg, or at least about 145 mg of the compound of formula (I), or a pharmaceutically acceptable salt thereof, on a free acid equivalent weight basis.

In certain embodiments, the compound of formula (I), or a pharmaceutically acceptable salt thereof, is administered orally to the subject. In certain embodiments, the compound of formula (I), or a pharmaceutically acceptable salt thereof, is administered orally to the subject daily. In certain embodiments, the compound of formula (I), or a pharmaceutically acceptable salt thereof, is administered orally to the subject once daily. In certain embodiments, the compound of formula (I), or a pharmaceutically acceptable salt thereof, is administered orally to the subject twice daily.

In certain embodiments, the compound of formula (0), or a pharmaceutically acceptable salt thereof, is administered orally to the subject once daily for 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 consecutive days. In certain embodiments, the compound of formula (I), or a pharmaceutically acceptable salt thereof, is administered orally to the subject once daily for 21 consecutive days.

In certain embodiments, the compound of formula (I), or a pharmaceutically acceptable salt thereof, is administered orally to the subject once daily for at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 consecutive days. In certain embodiments, the compound of formula (I), or a pharmaceutically acceptable salt thereof, is administered orally to the subject once daily for at least 21 consecutive days.

In certain embodiments, administering an effective amount of the compound of formula (I), or a pharmaceutically acceptable salt thereof, comprises administering to the subject about 10 mg to about 150 mg of the compound of formula (I), or a pharmaceutically acceptable salt thereof, on a free acid equivalent weight basis.

In certain embodiments, administering an effective amount of the compound of formula (I), or a pharmaceutically acceptable salt thereof, comprises administering orally to the subject about 10 mg to about 150 mg of the compound of formula (I), or a pharmaceutically acceptable salt thereof, on a free acid equivalent weight basis.

In certain embodiments, administering an effective amount of the compound of formula (I), or a pharmaceutically acceptable salt thereof, comprises administering orally to the subject about 10 mg to about 150 mg of the compound of formula (I), or a pharmaceutically acceptable salt thereof, on a free acid equivalent weight basis, daily.

In certain embodiments, administering an effective amount of the compound of formula (I), or a pharmaceutically acceptable salt thereof, comprises administering orally to the subject about 10 mg to about 150 mg of the compound of formula (I), or a pharmaceutically acceptable salt thereof, on a free acid equivalent weight basis, once daily.

In certain embodiments, administering an effective amount of the compound of formula (I), or a pharmaceutically acceptable salt thereof, comprises administering orally to the subject about 10 mg to about 150 mg of the compound of formula (I), or a pharmaceutically acceptable salt thereof, on a free acid equivalent weight basis, once daily for 21 consecutive days.

In certain embodiments, the subject is in a fasting state. In certain embodiments, the subject is not in a fasting state. In certain embodiments, administering an effective amount of the compound of formula (I), or a pharmaceutically acceptable salt thereof, comprises administering to the subject the effective amount about 1 hour before a meal or about 2 hours after a meal.

In certain embodiments, administering an effective amount of the compound of formula (I), or a pharmaceutically acceptable salt thereof, comprises administering to the subject about 10 mg to about 150 mg of the compound of formula (I), or a pharmaceutically acceptable salt thereof, on a free acid equivalent weight basis, about 1 hour before a meal or about 2 hours after a meal.

In certain embodiments, the subject has previously been administered at least one prior line of therapy. In certain embodiments, the subject has previously been administered fewer than five prior lines of therapy. In certain embodiments, the subject has previously been administered one, two, three, or 4 prior lines of therapy. In certain embodiments, the subject has not been administered a prior line of therapy.

In certain embodiments, the subject has previously been administered at least one and no more than 5 prior lines of therapy.

Prior lines of therapy include, but are not limited to, surgery, radiation therapy (e.g., external beam radiation therapy or internal radiation therapy), chemotherapy (e.g., alkylating agents, nitrosoureas, anti-metabolites, plant alkaloids and natural products, anti-tumor antibiotics, hormonal agents, and biological response modifiers), gene therapy, DNA therapy, viral therapy (e.g., oncolytic virus therapy), RNA therapy, adjuvant therapy, and immunotherapy (e.g., immune checkpoint inhibition, adoptive cell therapies, (e.g., tumor-infiltrating lymphocyte therapy, engineered T-cell receptor therapy, CAR T-cell therapy, natural killer cell therapy), or monoclonal antibodies).

In certain embodiments, the methods include administering an effective amount of a pharmaceutically acceptable salt of the compound of formula (I). In certain embodiments, the pharmaceutically acceptable salt is a potassium salt. In certain embodiments, the potassium salt is a hydrate. In certain embodiments, the potassium salt is a monohydrate.

In certain embodiments, the methods described herein further comprise administering an effective amount of a second therapeutic agent to the subject. In certain embodiments, the second therapeutic agent is selected from the group consisting of a checkpoint inhibitor, an EGFR inhibitor, an antiangiogenic agent, venetoclax, fluorouracil, and combinations thereof.

In certain embodiments, the second therapeutic agent is a checkpoint inhibitor In certain embodiments, the second therapeutic agent is a PD-1 or PD-L1 inhibitor. In certain embodiments, the second therapeutic agent is selected from the group consisting of nivolumab, pembrolizumab, atezolizumab, avelumab, durvalumab, cemiplimab, and dostarlimab.

In certain embodiments, the second therapeutic agent is an FGFR inhibitor. In some embodiments, the EGFR inhibitor is selected from the group consisting of erlotinib, gefitinib, afatinib, and osimertinib.

In certain embodiments, the second therapeutic agent is an antiangiogenic agent. In certain embodiments, the antiangiogenic agent is a VFGFR inhibitor.

In certain embodiments, the second therapeutic agent is a VEGFR inhibitor. In certain embodiments, the VEGFR inhibitor is selected from the group consisting of sunitinib, axitinib, lenvatinib, tivozanib, pazopanib, cabozantinib, and ramucirumab.

In another aspect, provided herein are methods of treating a solid tumor (e.g., a solid tumor/advanced solid tumor described herein) in a subject in need thereof, comprising administering to the subject any one of the pharmaceutical compositions described herein.

In another aspect, provided herein are methods of treating a blood cancer (e.g., a blood cancer described herein) in a subject in need thereof, comprising administering to the subject any one of the pharmaceutical compositions described herein.

In certain embodiments, the subject is a human. In certain embodiments, the subject is an adult human.

### EXAMPLES

In order that the disclosure may be more fully understood, the following examples are set forth. The synthetic and biological examples described in this application are offered to illustrate the compounds, pharmaceutical compositions, and methods provided herein and are not to be construed in any way as limiting their scope.

### Abbreviations

ACK ammonium-chloride-potassium
BID twice daily
FBS fetal bovine serum
FFPE formalin-fixed paraffin-embedded
HRP horseradish peroxidase
IHC immunohistochemistry
ISR integrated stress response
LN₂ liquid nitrogen
PBS phosphate buffered saline
PMN-MDSC polymorphonuclear myeloid-derived suppressor cell
PO oral administration
RBC red blood cell
RPMI Roswell Park Memorial Institute medium
SDS sodium dodecyl sulfate
STR short tandem repeat

### Example 1: Synthesis of 6-(3-((5-chloro-2-methoxypyridine)-3-sulfonamido)-2,6-difluorophenyl)-N-methylimidazo[1,5-a]pyrazine-1-carboxamide (Compound of Formula (I))

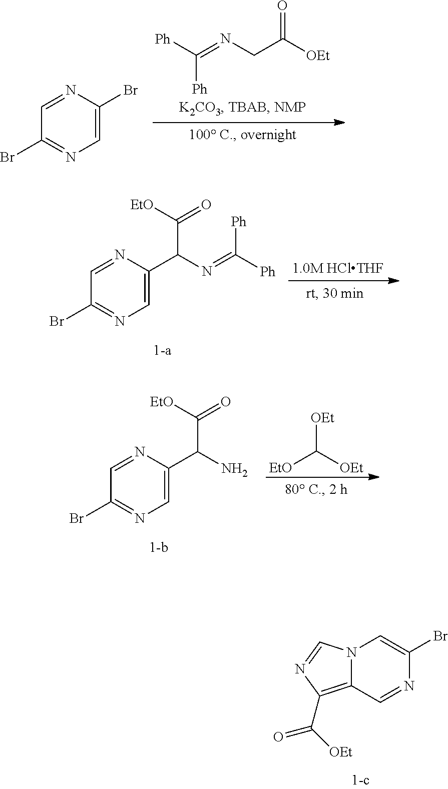 [SMILES: CCOC(=O)CN=C(c1ccccc1)c1ccccc1 ; Brc1cnc(Br)cn1 ; CCOC(=O)C(N=C(c1ccccc1)c1ccccc1)c1cnc(Br)cn1 ; CCOC(=O)C(N)c1cnc(Br)cn1 ; CCOC(OCC)OCC ; CCOC(=O)c1ncn2cc(Br)ncc12]

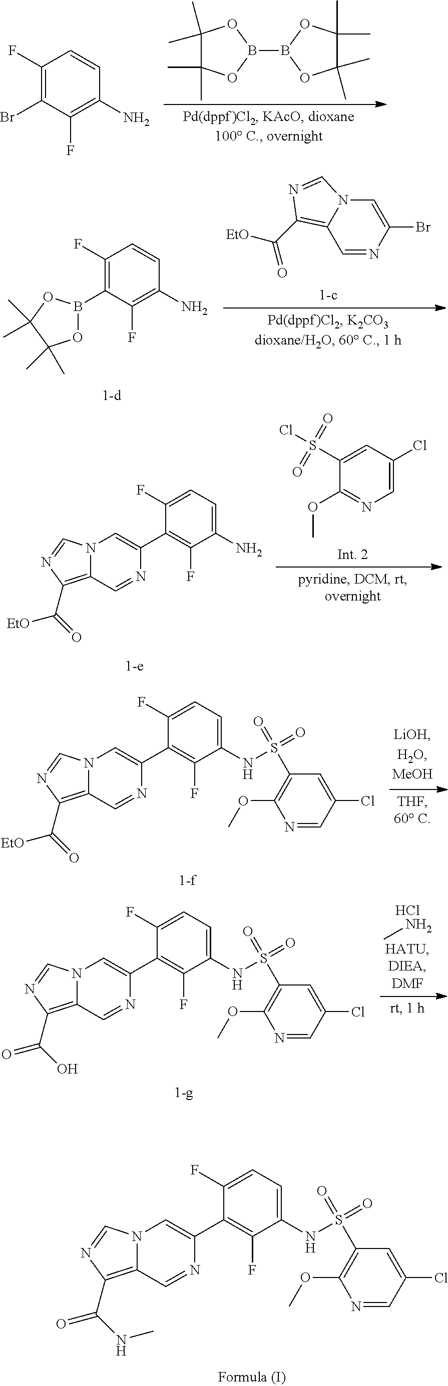

### Synthesis of 1-a: ethyl 2-(5-bromopyrazin-2-yl)-2-[(diphenylmethylidene) amino]acetate

2,5-Dibromopyrazine (10 g, 42 mmol, 1 equiv.), ethyl 2-[(diphenylmethylidene)amino]acetate (11.8 g, 44 mmol, 1.05 equiv.), tetrabutylammonium bromide (TBAB) (13.6 g, 42 mmol, 1 equiv.) and K₂CO₃ (17.4 g, 126 mmol, 3 equiv.) in (N-methyl-2-pyrrolidone) NMP (200 mL) were stirred overnight at 100° C. in an oil bath. The reaction mixture was cooled and filtered. The filtrate was diluted with 200 mL of water. The resulting solution was extracted with 2×200 mL of ethyl acetate and the organic layers combined. The resulting mixture was washed with 2×200 ml of water. The mixture was dried over anhydrous sodium sulfate and concentrated. The residue was applied to a silica gel column, eluting with ethyl acetate/petroleum ether (PE) (1/10). The collected fractions were combined and concentrated to give ethyl 2-(5-bromopyrazin-2-yl)-2-[(diphenylmethylidene)amino]acetate (8 g, 45% yield) as a yellow solid.

LCMS (ES, m/z): [M+H]⁺: 424

### Synthesis of 1-b ethyl 2-amino-2-(5-bromopyrazin-2-yl) acetate

Into a 250 mL round-bottom flask was placed ethyl 2-(5-bromopyrazin-2-yl)-2-[(diphenylmethylidene)amino]acetate (8 g, 18.8 mmol, 1 equiv.), tetrahydrofuran (THF) (10 mL) and HCl (aqueous, 1 M) (20 mL). The resulting solution was stirred for 30 min at 25° C. The solution formed was diluted with 50 mL of water and extracted with 2×50 mL of dichloromethane. The aqueous layers were adjusted to pH 8 with NH₃·H₂O and further extracted with 3×50 mL of dichloromethane. The combined organic layers were dried over anhydrous sodium sulfate and concentrated. Ethyl 2-amino-2-(5-bromopyrazin-2-yl)acetate (4.7 g, 96% yield) was isolated as a yellow solid which was used in next step directly without further purification.

LCMS (ES, m/z): [M+H]⁺: 260

### Synthesis of 1-c: ethyl 6-bromoimidazo [1,5-a]pyrazine-1-carboxylate

Into a 50 mL round-bottom flask was placed ethyl 2-amino-2-(5-bromopyrazin-2-yl) acetate (4.2 g, 0.02 mol, 1 equiv.) and triethyl orthoformate (20 mL). The resulting solution was stirred for 2 h at 80° C. in an oil bath. The reaction mixture was cooled, and the solids collected by filtration. Air drying gave ethyl 6-bromoimidazo [1,5-a]pyrazine-1-carboxylate (2.2 g, 50% yield) as a brown solid.

LCMS (ES, m/z): [M+H]⁺: 270

### Synthesis of 1-d: 2,4-difluoro-3-(4,4,5,5-tetramethyl-1,3,2-dioxaborolan-2-yl)aniline

3-Bromo-2,4-difluoroaniline (10 g, 48 mmol, 1 equiv.), [1,1′-Bis(diphenylphosphino)ferrocene]dichloropalladium(II) (Pd(dppf)Cl₂) (3.5 g, 4.8 mmol, 0.1 equiv.), bis(pinacolato)diboron (18.3 g, 72 mmol, 1.5 equiv.) and potassium acetate (KOAc) (14.2 g, 144.2 mmol, 3 equiv.) were dissolved in dioxane (240 mL). The resulting solution was stirred overnight at 100° C. in an oil bath. The reaction mixture was cooled, and the solids removed by filtration. The filtrate was concentrated and diluted with dichloromethane (DCM) (100 mL), then washed with 2×100 mL of water and 100 mL of brine. The organic phase was dried over anhydrous sodium sulfate and concentrated under reduced pressure. The residue was applied to a silica gel column, eluting with ethyl acetate/petroleum ether (1/10) 2,4-Difluoro-3-(4,4,5,5-tetramethyl-1,3,2-dioxaborolan-2-yl)aniline (8 g, 65% yield) was isolated as a yellow solid.

LCMS (ES, m/z): [M+H]⁺: 256

### Synthesis of 1-e: ethyl 6-(3-amino-2,6-difluorophenylimidazo[1,5-a]pyrazine-1-carboxylate

Ethyl 6-bromoimidazo[1,5-a]pyrazine-1-carboxylate (500 mg, 1.9 mmol, 1 equiv.), 2,4-difluoro-3-(4,4,5,5-tetramethyl-1,3,2-dioxaborolan-2-yl)aniline (708 mg, 2.8 mmol, 1.5 equiv.), bis(diphenylphosphino)ferrocene)palladium(II) dichloride (Pd(dppf)Cl₂) (135 mg, 0.2 mmol, 0.1 equiv.), K₂CO₃ (767 mg, 5.6 mmol, 3 equiv.) in dioxane (10 mL) and H₂O (2 mL) were stirred for 1 h at 60° C. in an oil bath. The reaction mixture was cooled, diluted with water (20 ml) and extracted with 3×20 mL of dichloromethane. The organic layers were dried over anhydrous sodium sulfate and concentrated. The residue was applied to a silica gel column and eluted with ethyl acetate/PE (1/2). Ethyl 6-(3-amino-2,6-difluorophenyl)imidazo[1,5-a]pyrazine-1-carboxylate (200 mg 34% yield) was isolated as a brown solid.

LCMS (ES, m/z): [M+H]⁺: 319

### Synthesis of 1-f: ethyl 6-[3-(5-chloro-2-methoxypyridine-3-sulfonamido)-2,6-difluorophenyl]imidazo[1,5-a]pyrazine-1-carboxylate

Ethyl 6-(3-amino-2, 6-difluorophenyl)imidazo[1,5-a]pyrazine-1-carboxylate (150 mg, 0.5 mmol, 1 equiv.) in DCM (5 mL) was treated with pyridine (186 mg, 2.3 mmol, 5 equiv.), then 5-chloro-2-methoxypyridine-3-sulfonyl chloride (137 mg, 0.6 mmol, 1.2 equiv.). The resulting solution was stirred overnight. The resulting mixture was concentrated and purified by Flash-Prep-HPLC with the following conditions: Column, WelFlash™ C18-I, Spherical C18 20-40 μm; mobile phase: 0.1% Formic Acid/5-70% acetonitrile (MeCN) over 15 min; Detector, 254 & 220 nm. Ethyl 6-[3-(5-chloro-2-methoxypyridine-3-sulfonamido)-2,6-difluorophenyl]imidazo[1,5-a]pyrazine-1-carboxylate (320 mg 97% yield) was isolated as a yellow solid.

LCMS (ES, m/z): [M+H]⁺: 524

### Synthesis of 1-g: 6-[3-(5-chloro-2-methoxypyridine-3-sulfonamido)-2,6-difluorophenyl]imidazo[1,5-a]pyrazine-1-carboxylic acid

Ethyl 6-[3-(5-chloro-2-methoxypyridine-3-sulfonamido)-2,6-difluorophenyl]imidazo[1,5-a]pyrazine-1-carboxylate (200 mg, 0.4 mmol, 1 equiv.), methanol (MeOH) (2 mL), THF (2 mL), H₂O (2 mL) and LiOH (27 mg, 1.1 mmol, 3 equiv.) were stirred for 1 h at 60° C. in an oil bath. After concentration, the crude product was purified by Flash-Prep-high performance liquid chromatography (HPLC) with the following conditions: Column, WelFlash™ C18-1, Spherical C18 20-40 μm; mobile phase: 5-60% acetonitrile (MeCN)/0.1% ammonia over 15 min; Detector, 254 nm. 6-[3-(5-Chloro-2-methoxypyridine-3-sulfonamido)-2,6-difluorophenyl]imidazo[1,5-a]pyrazine-1-carboxylic acid (170 mg, 90% yield) was isolated as a yellow solid.

LCMS (ES, m/z): [M+H]⁺: 496

### Synthesis of 6-[3-(5-chloro-2-methoxypyridine-3-sulfonamido)-2,6-difluorophenyl]-N-methylimidazo[1,5-a]pyrazine-1-carboxamide

6-[3-(5-Chloro-2-methoxypyridine-3-sulfonamido)-2,6-difluorophenyl]imidazo[1,5-a]pyrazine-1-carboxylic acid (170 mg, 0.3 mmol, 1 equiv) in N,N-dimethylformamide (DMF) (4 mL) was treated with diisopropylethylamine (DIEA) (133 mg, 1 mmol, 3 equiv.), methylamine hydrochloride (16 mg, 0.5 mmol, 1.5 equiv.) and 1-[Bis(dimethylamino)methylene]-1H-1,2,3-triazolo[4,5-b]pyridinium 3-oxide hexafluorophosphate (HATU) (195 mg, 0.5 mmol, 1.5 equiv.). The resulting solution was stirred for 1 hr, then concentrated. The crude product was purified by Flash-Prep-HPLC with the following conditions: Column, WelFlash™ C18-1, Spherical C18 20-40 μm, 120 g; mobile phase: 5-60% MeCN/0.1% formic acid over 20 min. 6-[3-(5-Chloro-2-methoxypyridine-3-sulfonamido)-2,6-difluorophenyl]-N-methylimidazo[1,5-a]pyrazine-1-carboxamide (43 mg, 25% yield) was isolated as an off-white solid.

liquid chromatography/mass spectrometry (LCMS) (ES, m/z): [M+H]⁺: 509

¹H nuclear magnetic resonance spectroscopy (NMR) (300 MHz, DMSO-d₆) δ 10.46 (s, 1H), 9.51 (d, J=1.6 Hz, 1H), 8.66 (d, J=5.0 Hz, 2H), 8.51 (d, J=2.6 Hz, H), 8.43 (d, J=4.9 Hz, 1H), 8.09 (d, J=2.6 Hz, 11H), 7.42 (td, J=8.8, 5.8 Hz, 1H), 7.25 (td, J=9.3, 1.4 Hz, 1H), 3.92 (s, 3H), 2.84 (d, J=4.7 Hz, 3H).

### Example 2: Preparation of the Compound of Formula (I) Potassium Salt

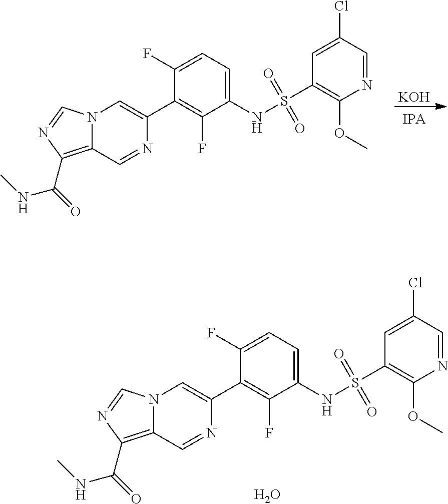

To a stirred mixture of 6-(3-((5-chloro-2-methoxypyridine)-3-sulfonamido)-2,6-difluorophenyl)-N-methylimidazo[1,5-a]pyrazine-1-carboxamide (as prepared in Example 1) in aqueous isopropyl alcohol (IPA) was slowly added 1.1 equivalents of an aqueous KOH solution and the solution heated. The resulting mixture was cooled, the solid collected, washed with IPA/water, and then dried under heat and vacuum to afford potassium ((5-chloro-2-methoxypyridin-3-yl)sulfonyl)(2,4-difluoro-3-(1-(methylcarbamoyl)imidazo[1,5-a]pyrazin-6-yl)phenyl)amide.

¹⁹F NMR (400 MHz, d6-DMSO): −129.62 and −127.72 ppm.

### Example 3: In Vivo Tumor Metastasis Studies

### Materials and Methods

### Tissue Culture, Tumor Engraftment, And Sample Collection

4T1 mouse breast cancer cell line was purchased from American Type Culture Collection (ATCC). Cells were free of human and animal pathogens as tested by h-IMPACT-I and IMPACT-III. Additionally, the genetic profile of the cells matched with the ECACC reference standard as tested by STR based DNA fingerprinting assay. Cells were maintained in RPMI 1640 with 10% FBS, and cultured up to 4 passages prior to implantation. Cells were harvested at ˜80% confluence, washed twice with RPMI 1640 without FBS and suspended in RPMI 1640 without FBS at 10×10⁶ cells/mL for implantation.

Female Balb/c mice (7-8 week of age) were implanted into the right 4^{th} mammary fat pad under Isoflurane anesthesia with 0.5×10⁶ 4T1 cells/mouse in a total volume of 50 mL on Day 0 Animals were randomized into treatment groups when the average tumor volume reached 50-100 mm³ on Day 6 post inoculation. Tumors were measured via caliper 2×/week. Tumor volume was calculated using the formula [tumor volume (mm³)=π/6×length×width²) Animals were sacrificed at either 7, 14, or 21 days post treatment via CO₂ asphyxiation.

Lungs were perfused with PBS through the heart to remove as much blood as possible prior to collection. Spleens were collected and weighed. The inferior right lobe of the lungs and 1/3 of the spleen were snap frozen in LN₂ for RNA gene expression analyses. The remaining lungs were collected with remaining spleen in RPMI medium+5% FBS on ice for flow cytometry. Separate animals were enrolled for lung IHC analyses. Following euthanasia of these animals, lungs were inflated with 10% neutral buffered formalin prior to formalin fixation for 48 hours and paraffin embedding.

### H&E Metastases Analysis and IHC

Multiple paraffin sections more than 200 μm apart were cut for each lung sample. Three sections were stained with H&E on Leica Autostainer while one adjacent section was immuno-stained for ASNS (Cell Signaling #92479) and PSAT1 (Proteintech #10501-1-AP) using the Bond Rx stainer (Leica Biosystems). Stained slides were digitized using Versa 200 slide scanner (Leica Biosystems). Images were analyzed using custom-written macros in ImageJ/FIJI (NIH) analyzing the relative proportion of tumor regions to the whole lung area. The tumor regions were transferred onto the images of immune-stained sections and intensities of ASNS and PSAT1 signals within these tumor regions were quantified. (Graphing and statistics were performed using Prism Graphpad).

### JESS

A small (lentil-sized) piece of frozen tumor was lysed in 500 μl of lysis buffer containing 2% SDS, 10% betamercaptoethanol, protease and phosphatase inhibitors (Roche, 04 693 159 001 and 04 906 837 001), and 1× Benzonase (Sigma, E1014-25KU). Each sample was homogenized using a polytron (PT 10-35 GT) for 30 seconds, and heated at 95° C. for 5 minutes. Protein concentrations were determined using a 660 nm Protein assay (Pierce, 22662), with the detergent compatibility reagent (Pierce, 22663), according to manufacturer instructions.

Protein samples were analyzed by JESS (ProteinSimple) using the 12-230 kDa separation module (ProteinSimple, SM-W004), and antibodies targeting PSAT1 (Proteintech, 10501-1-AP), ATF4 (AbCam, ab184909), TRIB3 (Proteintech, 66702-1), and ASNS (Proteintech, 14681-1-AP).

### Flow Cytometry

Tissue processing for single cell suspensions:
Spleen: Spleens were mashed through wetted 70 μm nylon strainer using syringe plunger. Strainers were rinsed with RPMI+5% FBS. Cells were pelleted and RBCs lysed with ACK lysis buffer (Gibco) per manufacturer's instructions. Cells were resuspended in RPMI+5% FBS and counted prior to plating for staining.
Lungs: Lungs were homogenized and enzymatically digested using a Mouse Tumor Dissociation Kit (miltenyi) per manufacturer's instructions. Cells were resuspended in RPMI+5% FBS and counted prior to plating for staining. During the digestion at 37° C., brefeldin A (Biolegend) was included in the enzyme mix to prevent export of cytokines.

1E6 cells per sample were plated in 96 well plates and extracellular staining was performed per standard procedures. Following extracellular staining, cells were fixed and permeabilized using FoxP3/Transcription Factor Staining Buffer Kit (Tonbo) and intracellular staining was performed per standard procedures. The following antibodies were used.

TABLE 1
T-cell panel:

| | | | | | Dilution |
|---|---|---|---|---|---|
| Target | Vendor | Clone | Catalogue | Fluorochrome | Factor |
| brilliant stain | BD | N/A | 556385 | N/A | 5 |
| buffer plus | | | | | |
| CD45 | Biolegend | 30-F11 | 103138 | BV510 | 250 |
| CD4 | BD | GK1.5 | 563790 | BUV395 | 200 |
| CD8 | BD | 53-6.7 | 612898 | BUV805 | 150 |
| CD3 | BD | 17A2 | 557869 | APC-Fire810 | 100 |
| IL2 (intracellular) | BD | JES6.5H4 | 566363 | BV750 | 100 |
| Ki67 (intracellular) | eBioscience | SolA15 | 58-5698-82 | AF532 | 100 |
| Tbet (intracellular) | Biolegend | 4B10 | 644810 | PE | 100 |
| Granzyme B | eBioscience | NG2B | 35-8898-82 | PE-Cy5.5 | 100 |
| (intracellular) | | | | | |
| CD98 | Biolegend | Rl388 | 128212 | APC | 80 |

TABLE 2
Myeloid panel:

| | | | | | Dilution |
|---|---|---|---|---|---|
| Target | Vendor | Clone | Catalogue | Fluorochrome | Factor |
| CD45 | BD | 30-F11 | 564279 | BUV395 | 1/250 |
| CD11b | BD | M1/70 | 741242 | BUV563 | 1/250 |
| Ly6G | Biolegend | 1A8 | 127645 | BV785 | 1/200 |
| MHCII (I-A/I-E) | BD | M5/114 | 748844 | BUV805 | 1/1000 |
| CD98 | eBioscience | RL388 | 12-0981-83 | PE | 1:100 |

Following staining, flow cytometry was performed on a Cytek Aurora cytometer and analyzed using FlowJo software.

### Gene Expression Analysis

Mouse tumor and spleen tissues were snap frozen and stored in −80° C. freezer. The frozen tissue was soaked in 10× volume of the RNAlater®-ICE (ThermoFisher) at −20° C. for >16 hr. Then the tissue was transferred into M tube (Miltenyi Biotech) containing RLT lysis buffer (Qiagen) and homogenized by gentleMACS Dissociators with program of RNA_02 (Miltenyi Biotech). Total RNA was isolated from homogenized tissue using the RNeasy Plus Mini Kit (Qiagen) RNA concentration was determined by Epoch Microplate Spectrophotometer (Biotek).

A custom-made 77-plex mouse Immune QuantiGene Assay kit (ThermoFisher Scientific) using a bead-based multiplex assay were used to measure the expression of 69 immune genes, 4 stress genes plus 4 reference genes with Luminex xMAP 200 (ThermoFisher Scientific) according to the manufacturer's instructions.

### Ex Vivo Analysis of Isolated Immune Cells

Single cell suspensions from mouse bone marrow were prepared. Cells were cultured in RPMI complete medium supplemented with GMCSF & IL6 (10 μg/mL) for 4 days to differentiate cells into PMN-MDSCs. T-cells were purified from mouse spleens with EasySep Mouse T-cell isolation kit per manufacturer's instructions Purified T-cells cells were then stained with CellTrace Violet dye (thermo) per manufacturer's instructions. Differentiated MDSCs and purified T-cells were co-cultured in tryptophan-depleted medium+/−HC-7366 (0.1 μM) for 6 h, at a ratio of 1:8 MDSCs:T-cells. Proliferation of T-cells was measured by dilution of CTV via flow cytometry on a BD LSR Fortessa.

### Imaging

Slides were cut from FFPE lung samples. Slides were deparaffinized, rehydrated and heat mediated antigen retrieval was conducted with AR6 buffer (Akoya). Slides were blocked with Roche diagnostics antibody diluent (Fisher) and stained with primary antibodies for 1 hr at 110 rpm. HRP-conjugated secondary antibodies were added for 10 mins at 110 rpm followed by 10 mins with OPAL detection dye (Akoya). For multiplex staining, this process was repeated starting at the antigen retrieval stage. Multispectral Images were captured on the Polaris imaging system (Akoya) and spectral unmixing and cell segmentation was performed using Inform Tissue Finder software (Akoya). Imaging data was converted into .csv files and imported into Flowjo for immune phenotyping.

Primary antibodies used were rat anti-mouse CD4 (clone 4SM95, Thermo 14-9766-82-82, paired with Opal 620), rabbit anti-mouse Ki67 (clone SP6, Thermo MA5-14520, paired with Opal 780), rabbit anti-mouse CD8a (clone D4W2Z, Cell Signaling 98941, paired with Opal 520). Rabbit anti-mouse S1008A (Polyclonal, Proteintech 15792-1-AP, paired with Opal 520), rabbit anti-mouse S1009A (Polyclonal, Proteintech 26992-1-AP, paired with Opal 570), rabbit anti-mouse CD11b (clone EPR1344, Abcam 133357, paired with Opal 690).

### Results

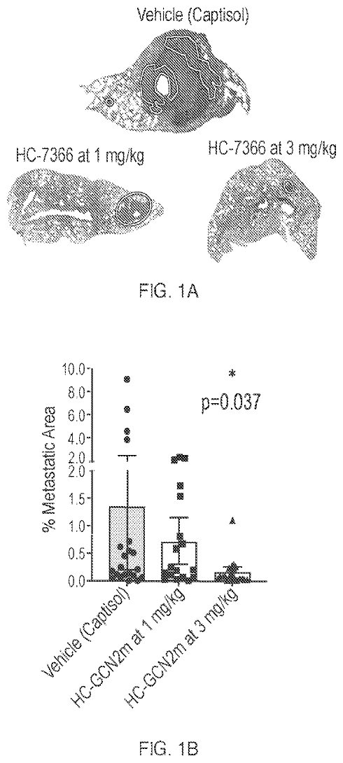

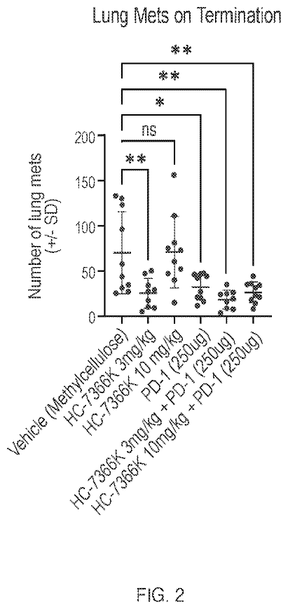

The 4T1 murine orthotopic allograft tumor model was used to analyze the effects of HC-7366 on tumor metastasis. Treatment of mice with 30 mg/kg of HC-7366 yielded a significant reduction in overall metastatic area in lung samples collected from allografted mice (FIGS. 1A-1B). A combination therapy of HC-7366 with anti-PD-1 therapy also showed a significant reduction in the number of lung metastases (FIG. 2).

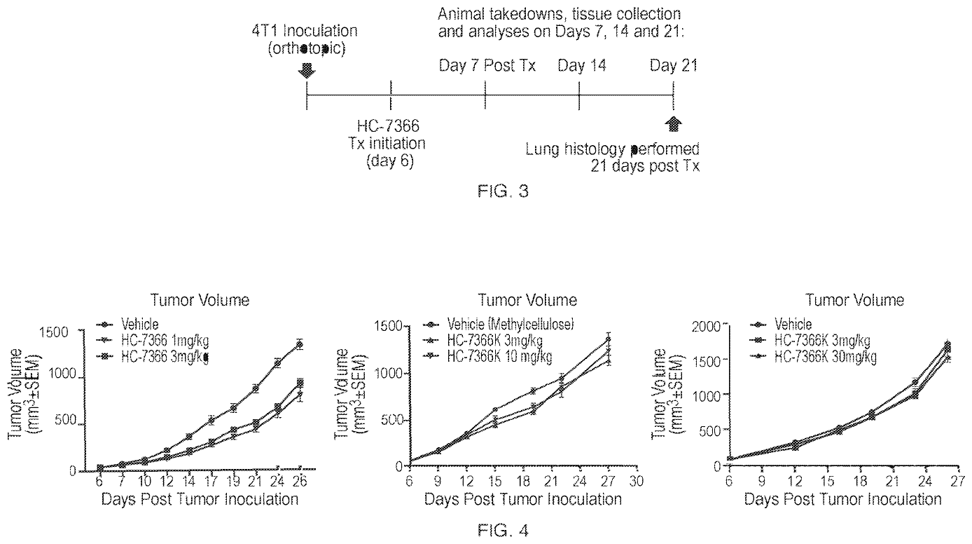

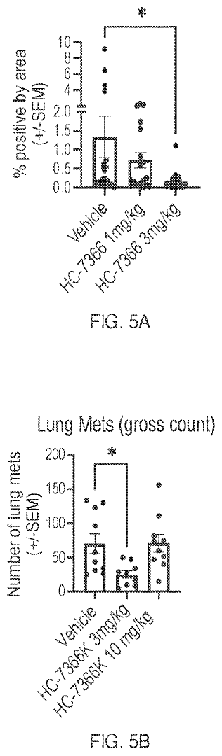

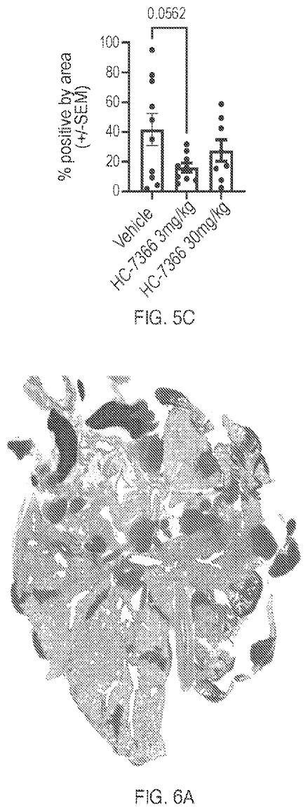

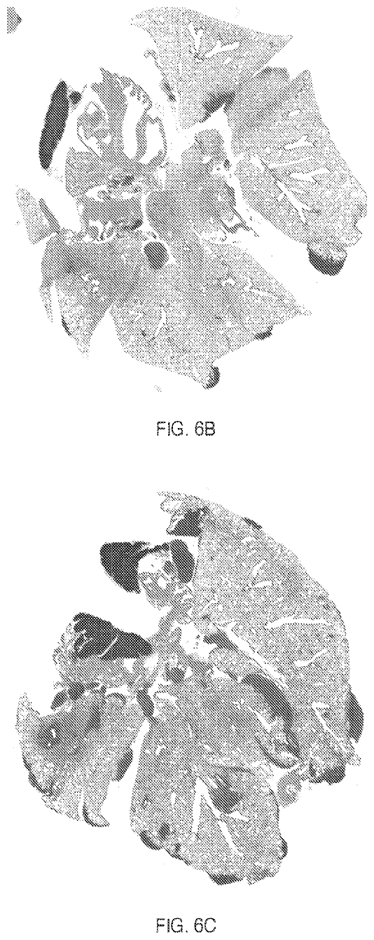

FIG. 3 shows a schematic of the experimental time course in which 4T1 breast cancer cells were inoculated orthotopically into the mammary fat pad of female BALB/c mice. Animals were randomized for treatment when tumors reached an average of 50-100 mm³. Treatment was administered PO, BID. Animals were sacrificed for analyses at 7, 14, and 21 days post treatment initiation. Three independent studies were conducted. HC-7366 showed little to no efficacy on primary 4T1 tumors across studies (FIG. 4) but showed a consistent anti-metastatic effect in lungs of tumor bearing mice administered 1-3 mg/kg HC-7366 (FIGS. 5A-5C). Lung samples were collected 21 days following treatment initiation (FIGS. 6A-6C).

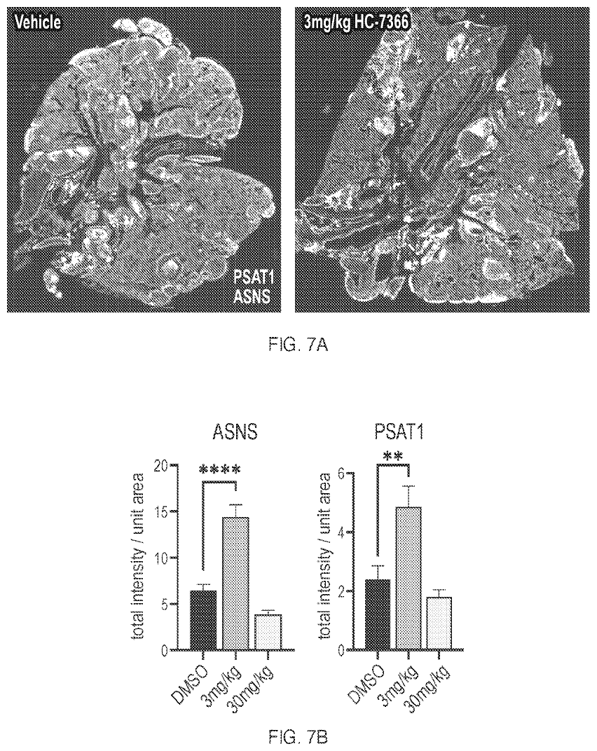

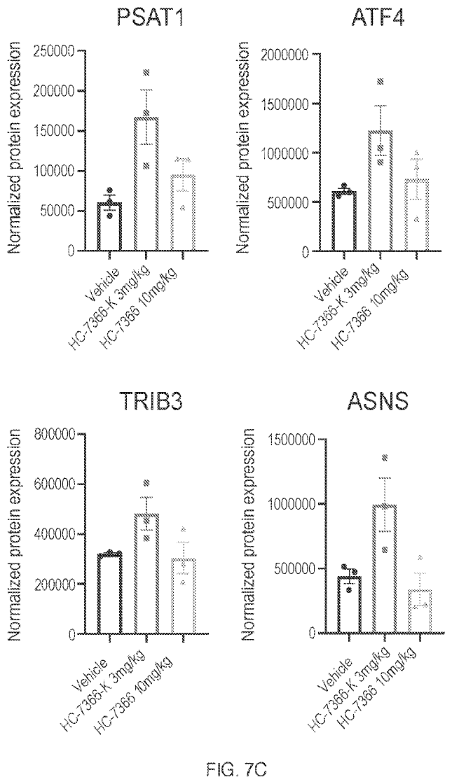

Lungs from 4′11 tumor-bearing mice were collected, fixed, and paraffin embedded 21 days post HC-7366 treatment initiation. IHC was performed to measure expression of the ISR-induced amino acid biosynthesis proteins asparagine synthetase (ASNS) and phosphoserine aminotransferase 1 (PSAT1) (FIG. 7A). Significant increases in both targets were observed with 3 mg/kg HC-7366 treatment (FIG. 7B). Primary 4T1 tumors collected 7 days post treatment initiation were homogenized and measured via JESS for ISR-induced proteins. Increases in PSAT1, ATF4, TRIB3, and ASNS were observed with 3 mg/kg HC-7366 treatment (FIG. 7C).

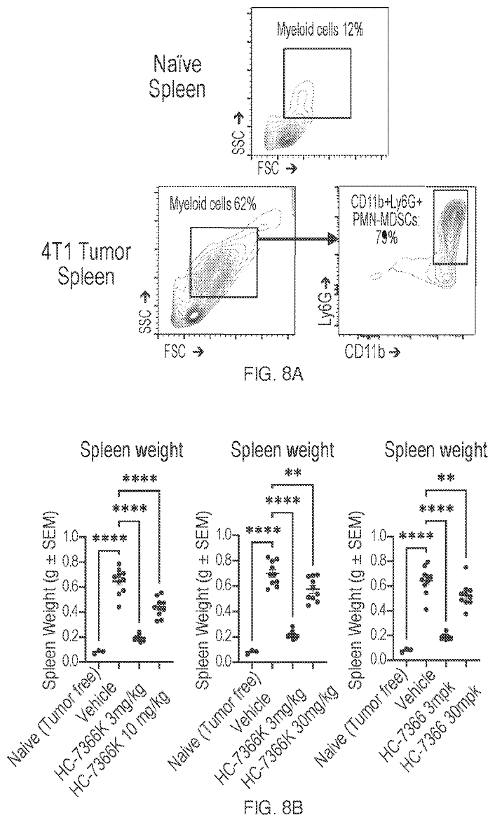

The effects of HC-7366 treatment on the spleens of 4T1 allografted mice were analyzed. Spleens in 4T1 tumor-bearing mice are enriched for CD11b+Ly6G+ PMN-MDSCs (FIG. 8A). MDSC expansion in 4T1 mice dramatically increases spleen weight compared to naïve mice. Treatment with HC-7366 significantly decreases splenomegaly, especially at the activating 3 mg/kg dose level (FIG. 8B, graphs represent 3 independent studies).

Gene expression analysis was performed to measure the effects of HC-7366 on the activation of myeloid cells. RNA isolated from tumors and spleens of HC-7366 treated 4T1 mice was analyzed for gene expression via Quantigene (Day 21 of Tx). While tumor tissue did not show consistent effects, the MDSC-enriched spleens showed consistent increases in immune activation transcripts and reductions in inhibitory transcripts with 3 mg/kg HC-7366 (FIG. 9).

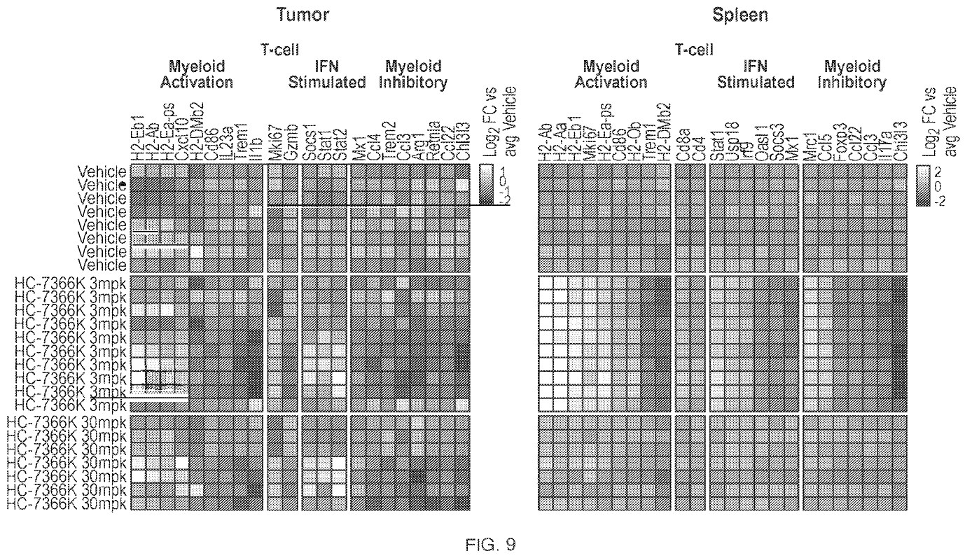

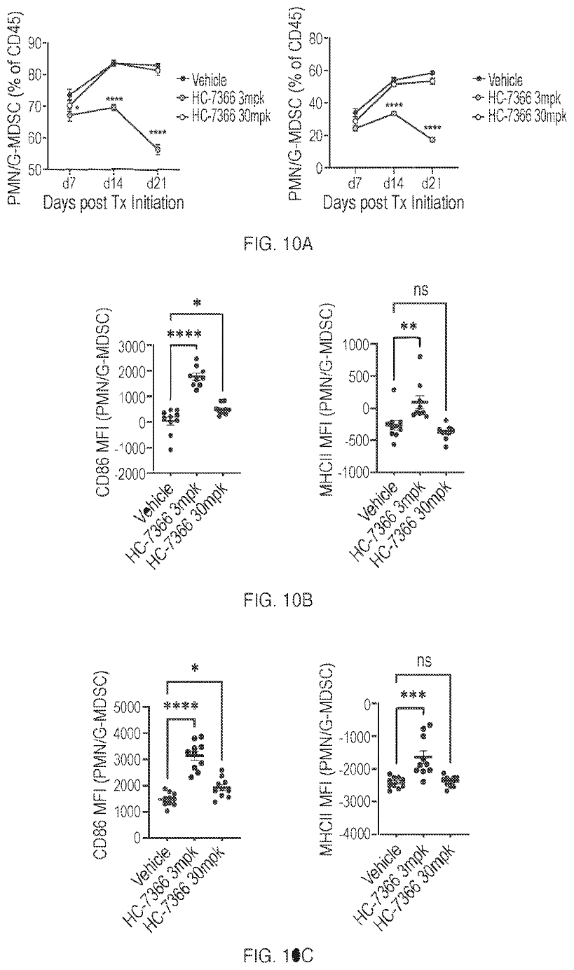

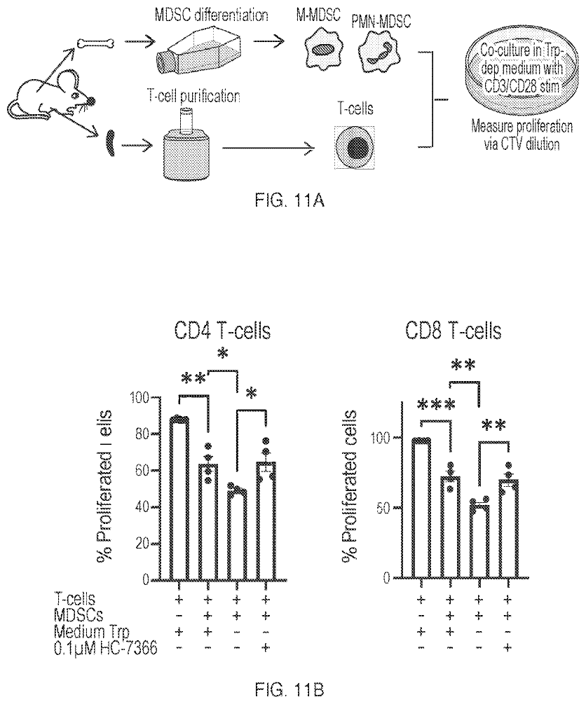

The effects of HC-7366 on the presence of MDSCs in allografted mice was next analyzed. Single cell suspensions from spleens and lungs of HC-7366 treated 4T1 mice were evaluated via multi-color flow cytometry. MDSC frequency in both organs was significantly reduced with 3 mg/kg HC-7366 treatment (FIG. 10A). The activation and co-stimulation markers MHCII and CD86 were significantly increased on MDSCs in the spleen and lungs with 3 mg/kg HC-7366 treatment (Day 14) (FIGS. 10B-10C). Furthermore, the activity of HC-7366 treated MDSCs was tested ex vivo. MDSCs differentiated from mouse bone marrow were co-cultured with purified T-cells in tryptophan-depleted medium with or without 0.1 μM HC-7366 as depicted in FIG. 11A. Proliferation of T-cells was measured by CTV dilution using flow cytometry. HC-7366 treatment significantly rescued T-cell proliferation in the presence of BM-derived MDSCs (FIG. 11B).

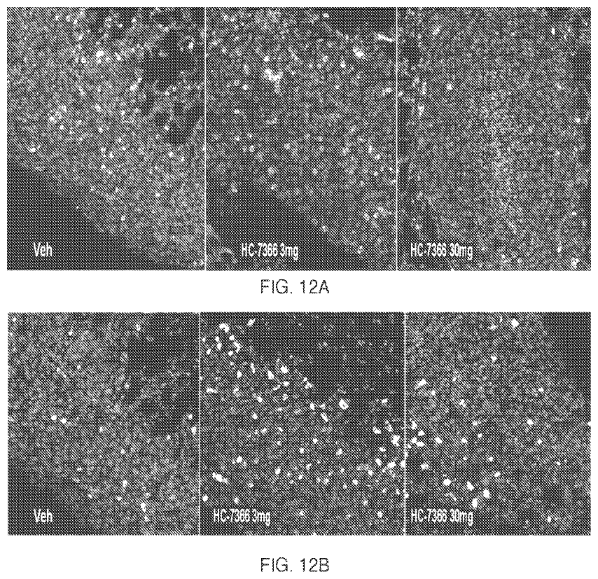

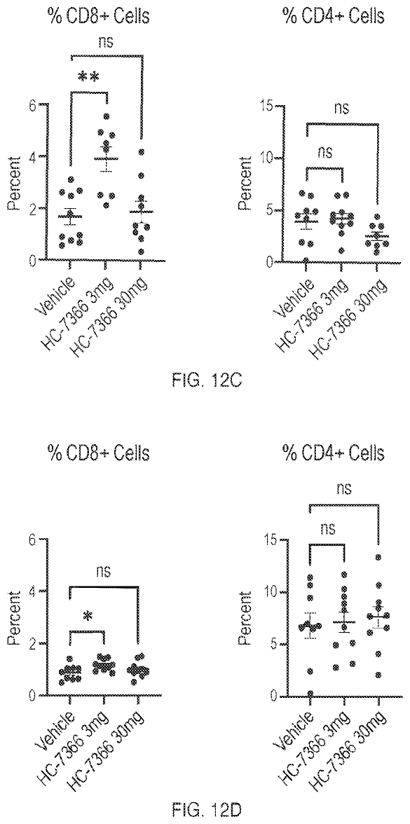

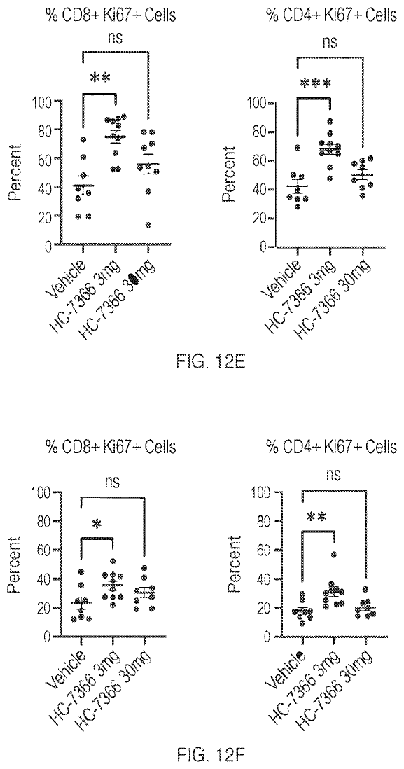

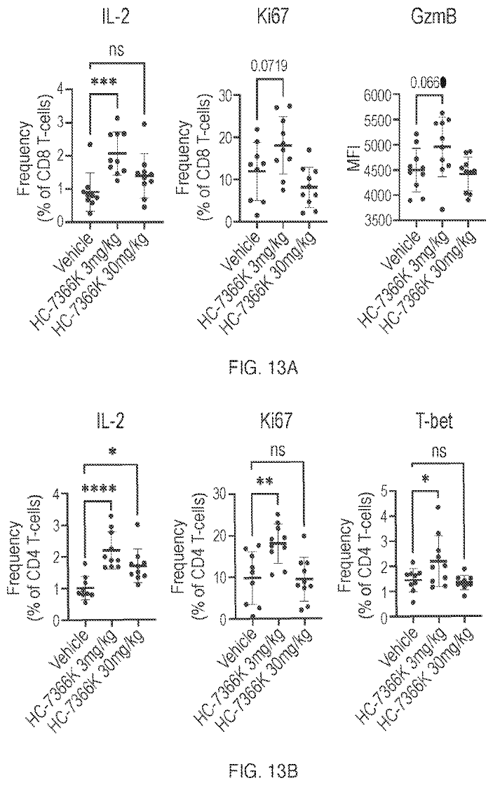

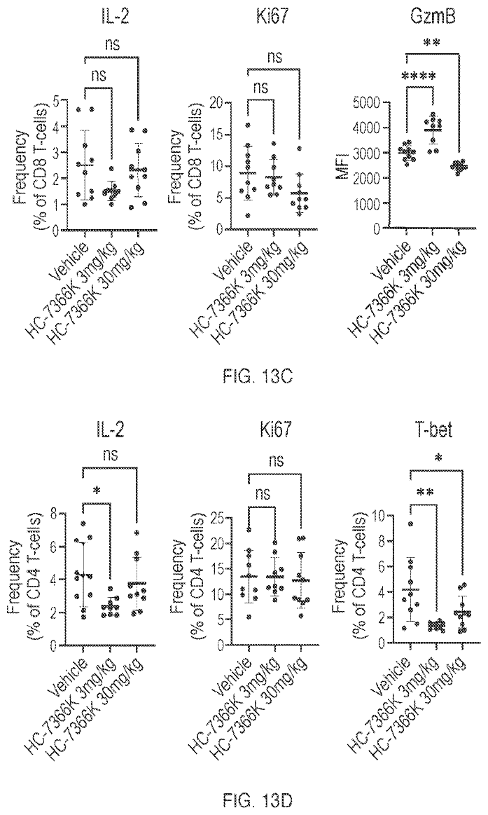

Imaging was used to measure the effects of HC-7366 treatment on T cell proliferation in metastases of 4T1 allografted mice. Lung samples were stained for CD4 (FIG. 12A) and CD8 (FIG. 12B). CD8 T-cells were significantly increased in metastatic lesions and to a lesser degree in normal tissue. Both CD4 and CD8 T-cells showed significantly increased Ki67 expression, which was greater in metastatic lesions than in normal tissue (FIGS. 12C-12F). Single cell suspensions from lungs and spleens of HC-7366 treated 4T1 mice were evaluated via intracellular flow cytometry (Day 21 of Tx). CD4 and CD8 T-cells showed significantly increased activation markers in lungs (FIGS. 13A-13B), but not in spleens (FIGS. 13C-13D).

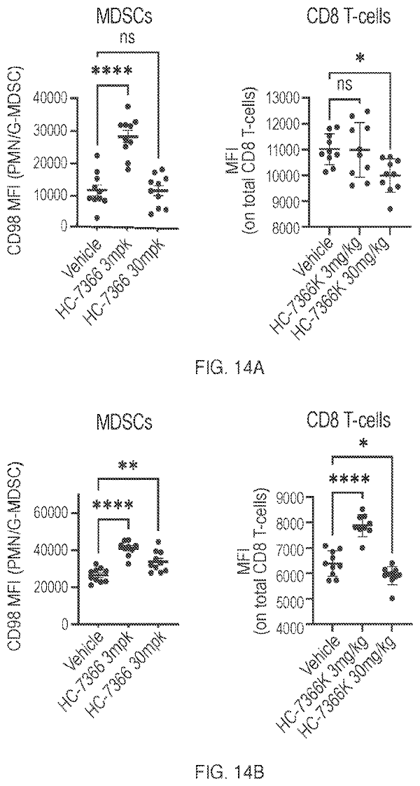

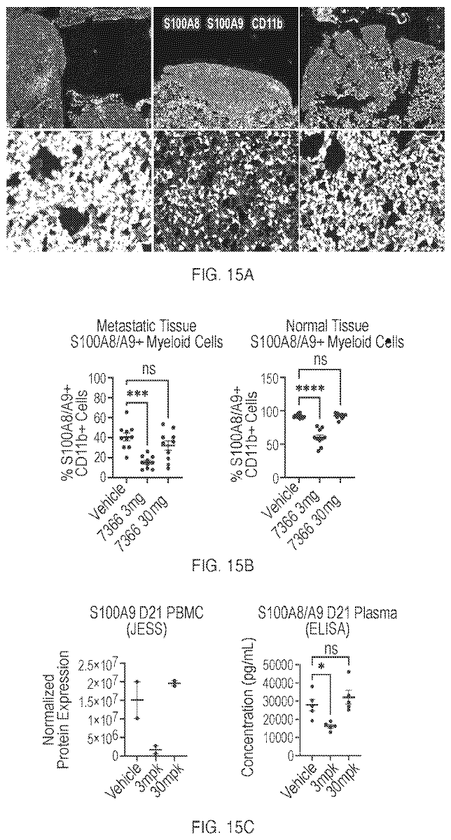

Single cell suspensions from lungs and spleens of 4T1 mice were stained for the ATF4-induced amino acid transporter CD98. 3 mg/kg HC-7366 treatment significantly increased expression of CD98 on MDSCs but not on CD8 T-cells in the lungs (FIG. 14A), while both cell subsets showed increased CD98 expression in the spleen (FIG. 14B). The levels of the inflammatory proteins S100A8 and S100A9, which are important for MDSC recruitment, expansion, and suppressive activity, were measured in normal lung tissue and lung metastases, as well as in PBMCs and plasma of 4T1 allografted mice, with significant reductions found in each case (FIGS. 15A-15C).

### INCORPORATION BY REFERENCE

This application refers to various issued patents, published patent applications, journal articles, and/or other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed:

1. A method of inhibiting myeloid-derived suppressor cells (MDSCs) in a subject in need thereof, comprising administering to the subject a therapeutically effective amount of a compound of formula (I) 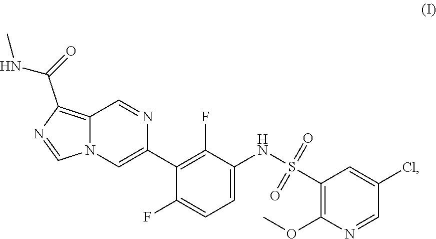 [SMILES: CNC(=O)c1ncn2cc(-c3c(F)ccc(NS(=O)(=O)c4cc(Cl)cnc4OC)c3F)ncc12]
or a pharmaceutically acceptable salt thereof.

2. The method of claim 1, wherein the method activates the integrated stress response (ISR) in the MDSCs.

3. A method of treating metastases in a subject in need thereof, comprising administering to the subject a therapeutically effective amount of a compound of formula (I) 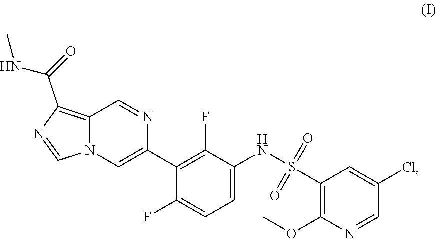 [SMILES: CNC(=O)c1ncn2cc(-c3c(F)ccc(NS(=O)(=O)c4cc(Cl)cnc4OC)c3F)ncc12]
or a pharmaceutically acceptable salt thereof.

4. A method of reducing tumor volume of metastases in a subject in need thereof, comprising administering to the subject a therapeutically effective amount of a compound of formula (I) 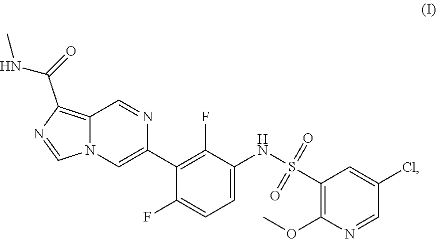 [SMILES: CNC(=O)c1ncn2cc(-c3c(F)ccc(NS(=O)(=O)c4cc(Cl)cnc4OC)c3F)ncc12]
or a pharmaceutically acceptable salt thereof.

5. The method of claim 3 or 4, wherein the metastases are present in one or more sites selected from the group consisting of the subject's lung, liver, bone, brain, peritoneum, adrenal gland, skin, and muscle.

6. The method of any one of claims 3-5, wherein the metastases have metastasized from a primary tumor.

7. The method of claim 6, wherein the primary tumor is a solid tumor.

8. The method of claim 6 or 7, wherein the primary tumor is selected from the group consisting of a breast cancer, non-small cell lung cancer (NSCLC), head and neck cancer, colorectal cancer, ovarian cancer, and hepatocellular carcinoma.

9. The method of any one of claims 6-8, wherein the primary tumor is a breast cancer.

10. The method of any one of claims 1-9, wherein the compound of formula (I), or a pharmaceutically acceptable salt thereof, is administered once daily.

11. The method of any one of claims 1-10, wherein the compound of formula (I), or a pharmaceutically acceptable salt thereof, is administered in an amount of about 10 mg to about 75 mg once daily.

12. The method of any one of claims 1-10, wherein the compound of formula (I), or a pharmaceutically acceptable salt thereof, is administered in an amount of about 75 mg to about 150 mg once daily.

13. The method of any one of claims 1-12, wherein the compound of formula (I), or a pharmaceutically acceptable salt thereof, is administered over a period of 2 years.

14. The method of any one of claims 1-13, wherein the compound of formula (I), or a pharmaceutically acceptable salt thereof, is administered orally.

15. The method of any one of claims 1-14, wherein the compound of formula (I), or a pharmaceutically acceptable salt thereof, is administered in the form of a capsule.

16. The method of any one of claims 1-15, wherein the method reduces the inhibition of T cells in the subject relative to a subject receiving placebo.

17. The method of any one of claims 1-16, wherein the method increases the activation of the ISR in MDSCs in the subject relative to a subject receiving placebo.

18. The method of any one of claims 1-17, wherein the method decreases the frequency of MDSCs in the lungs, spleen, and/or blood of the subject relative to a subject receiving placebo.

19. The method of any one of claims 1-18, wherein the method increases MDSC expression of CD86 and/or MHCII in the subject relative to a subject receiving placebo.

20. The method of any one of claims 1-19, wherein the method reduces S100 levels in CD11b+ cells in the subject relative to a subject receiving placebo.

21. The method of any one of claims 1-20, wherein the method reduces S100 levels in peripheral blood mononuclear cells (PBMCs) and/or plasma in the subject relative to a subject receiving placebo.

22. The method of any one of claims 1-21, wherein the method increases IL-2, Ki67, and/or T-bet on CD4 T-cells in the subject relative to a subject receiving placebo.

23. The method of any one of claims 1-22, wherein the method increases IL-2, Ki67, and/or T-bet on CD8 T-cells in the subject relative to a subject receiving placebo.

24. The method of any one of claims 1-23, wherein the method increases ISR pathway activation in the subject in MDSCs to a greater extent than in T-cells.

25. The method of any one of claims 1-24, wherein the compound of formula (I), or a pharmaceutically acceptable salt thereof, is administered as a pharmaceutically acceptable salt of the compound of formula (I).

26. The method of claim 25, wherein the pharmaceutically acceptable salt is a potassium salt.

27. The method of claim 26, wherein the potassium salt is a potassium salt hydrate.

28. The method of claim 27, wherein the potassium salt hydrate is a potassium salt monohydrate.
